(19) United States
(12) Patent Application Publication
(10) Pub. No.: US 2023/0207128 A1
(43) Pub. Date: Jun. 29, 2023

(54) PROCESSING ENCRYPTED DATA FOR ARTIFICIAL INTELLIGENCE-BASED ANALYSIS

(71) Applicant: AIONCO, Inc., Menlo Park, CA (US)

(72) Inventors: Stanley Chang, Cupertino, CA (US); Wendy Kewen Wang, Menlo Park, CA (US)

(21) Appl. No.: 18/147,680

(22) Filed: Dec. 28, 2022

Related U.S. Application Data
(60) Provisional application No. 63/294,796, filed on Dec. 29, 2021.
(60) Provisional application No. 63/330,728, filed on Apr. 13, 2022.

(51) Int. Cl. G16H 50/20; G06N 20/00; H04L 9/00; H04L 9/06; G06F 21/62; G06F 21/60

(52) U.S. Cl. CPC G16H 50/20; G06F 21/602; G06F 21/6245; G06N 20/00; H04L 9/008; H04L 9/0618

(57) ABSTRACT

Introduced here is an approach for managing errors generated during artificial intelligence-based analysis encrypted data. As an illustrative example, a computing system can may be configured to generate, train, and/or implement machine learning (ML) models to detect or predict aspects of one or more types of cancer based on homomorphically encrypted patient health data. The computing system may selectively identify timing for implementing a noise management mechanism during the data processing for the ML models.

### CROSS-REFERENCE TO RELATED APPLICATION(S)

The present application claims the benefit of U.S. Provisional Pat. Application No. 63/294,796 filed Dec. 29, 2021 and U.S. Provisional Pat. Application No. 63/330,728 filed Apr. 13, 2022, both of which are incorporated herein by reference in their entirety.

### TECHNICAL FIELD

Various implementations concern computer programs and associated computer-implemented techniques for processing encrypted data.

### BACKGROUND

Growth in computing technologies is creating new applications for identifying and learning previously unknown or undetected patterns and behaviors. For example, machine learning (ML), data mining, and other forms of artificial intelligence-based technology is being used for detecting, predicting, and recognizing new information, such as physiological conditions, individual behavior, social/group behavior, complex relationships between entities, and the like.

In some applications, the potential benefits of the technological growth must be weighed against the desired restrictions on access to related information. For example, the potential to find new ways to detect and treat diseases, such as cancer, cannot overwhelm the need to protect personal information and other types of sensitive data (e.g., healthcare data). Conventional methods of preserving privacy and security for such applications often require additional processes, machines, or algorithms that are commercially impractical and/or technologically unreliable (e.g., prone to errors).

As an illustrative example, patients genetic information may be analyzed to assess onset or risk of various forms of cancer. Genes are pieces of deoxyribonucleic acid (DNA) inside cells that indicate how to make the proteins that the human body needs to function. At a high level, DNA serves as the genetic “blueprint” that governs operation of each cell. Genes can not only affect inherited traits that are passed from a parent to a child, but can also affect whether a person is likely to develop diseases like cancer. Changes in genes -also called “mutations” - can play an important role in the physiological conditions of the human body, such as in the development of cancer. Accordingly, genetic testing may be leveraged to detect such physiological conditions or likely onsets thereof.

The term “genetic testing” may be used to refer to the process by which the genes or portions of genes of a person are examined to identify mutations. There are many types of genetic tests, and new genetic tests are being developed at a rapid pace. While genetic testing can be employed in various contexts, it may be used to detect mutations that are known to be associated with cancer.

Genetic testing could also be employed as a means for addressing or treating the physiological condition. For example, after a person has been diagnosed with cancer, a healthcare professional may examine a sample of cells to look for changes in the genes in tracking the progress of the cancer, the treatment, etc. These changes may be indicative of the health of the person (and, more specifically, progression/regression of the cancer). Insights derived through genetic testing may provide information on the prognosis, for example, by indicating whether treatment has been helpful in addressing the mutation.

Implementing computing technologies for the genetic testing may yield valuable insights. For example, artificial intelligence and machine-learning technologies may be leveraged to analyze DNA information for detecting and/or addressing cancers or potential onset of cancers. However, the magnitude of the DNA information, the large number of potential mutations, large number of samples, and other similar factors often negatively impact the effectiveness, the accuracy, and the practicality in leveraging such computing technologies for the genetic testing and the corresponding privacy protection.

### BRIEF DESCRIPTION OF THE DRAWINGS

FIGS. 1A and 1B show example operating environments of a computing system including a genetic information processing system in accordance with one or more implementations of the present technology.

FIG. 2 shows an example data processing formats for the genetic information processing system in accordance with one or more implementations of the present technology.

FIG. 3 shows example expected phrases in accordance with one or more implementations of the present technology.

FIG. 4 shows example derived phrases in accordance with one or more implementations of the present technology.

FIG. 5 shows an example analysis template in accordance with one or more implementations of the present technology.

FIG. 6 shows an example control flow diagram illustrating the functions of the system in accordance with one or more implementations of the present technology.

FIG. 7 shows an example implementation of the noise mitigation mechanism of FIG. 1 in accordance with one or more implementations of the present technology.

FIG. 8 shows a flow chart of a method for processing and refining DNA-based text data for cancer analysis in accordance with one or more implementations of the present technology.

FIG. 9 shows a flow chart of an example method for configuring the cancer analysis to process encoded data with noise management in accordance with one or more implementations of the present technology.

FIG. 10 shows a flow chart of an example method for implementing the trained model with noise management in accordance with one or more implementations of the present technology.

FIG. 11 is a block diagram illustrating an example of a system in accordance with one or more implementations of the present technology.

Various features of the technology described herein will become more apparent to those skilled in the art from a study of the Detailed Description in conjunction with the drawings. Various implementations are depicted in the drawings for the purpose of illustration. However, those skilled in the art will recognize that alternative implementations may be employed without departing from the principles of the technology. Accordingly, although specific implementations are shown in the drawings, the technology is amenable to various modifications.

### DETAILED DESCRIPTION

Genetic testing may be beneficial for diagnosing and treating cancer. For example, identifying mutations that are indicative of cancer can help (1) healthcare professionals make appropriate decisions, (2) researchers to direct their investigations, and (3) precision medicine to design better therapies. However, discovering these mutations tends to be difficult, especially as the number of cancers of interest (and thus, corresponding data) increases.

While computer-aided detection (CADe) and computer-aided diagnostic (CADx) processing systems may be used to analyze the genetic testing data, conventional approaches still face several drawbacks due to the overwhelming number of computations required for such analysis. For example, conventional systems may identify a number of molecular positions (e.g., target analysis locations) and combinations that may inefficient, ineffective, inaccurate, or otherwise impractical to process. Moreover, such deficiencies become even more problematic when the system is tasked with reviewing the genetic information of tens, hundreds, or thousands of patients. In other words, even if a conventional system is able to comprehensively analyze the genetic information of a single patient, reviewing the genetic information of tens, hundreds, or thousands of patients during actual deployment becomes impractical due to the processing delays and inaccuracies.

In further complicating the matter, the processed genetic information is highly sensitive and private. As such, the patient, the healthcare provider, and the data processing entity have strong interests in protecting the data against unauthorized access. While typical approach may be to encrypt the sensitive data, encryptions add further complexities and increase the processing burdens of the overall processing mechanism. As such, genetic testing using encrypted DNA data becomes even less practical for conventional approaches.

Introduced here is first an approach that can be implemented by a computing system to predict and/or diagnose in an improved manner. Implementations of the present technology can include the computing system processing the genetic information as relatively simple/smaller computer-readable data, such as text strings (simpler/smaller in comparison to, e.g., image data). Using the textual representations, the computing system can identify specific text patterns, such as unique segments of repeated characters (e.g., tandem repeats (TRs) corresponding to sequences of two or more DNA bases that are repeated numerous times in a head-to-tail manner on a chromosome), phrases surrounding the unique segments, and derivations/mutations thereof, used to analyze nucleic acid sequences (or simply “sequences”). In some implementations, the computing system can focus on the unique phrases and/or derivations thereof in characterizing and/or recognizing one or more types of cancer. In some implementations, the computation system can select features from the phrases/derivations and may ignore other portions of the overall text string or sequence, thereby reducing the overall computations in developing, training, and/or applying a machine learning (ML) model or other artificial intelligence mechanisms. While implementation of the approach may result in improvements across different aspects of mutation discovery, there are several notable improvements worth mentioning.

Advantageously, the approach allows models to be trained (and diagnoses to be predicted by those trained models) in a more time- and resource-efficient manner as the number of features considered by the computing system may be reduced (e.g., from tens of thousands of nucleotide locations to several thousand nucleotide locations). For a given type of cancer, the computing system can reduce an expanded feature set that is discovered through examination of training of genetic information through ML, so as to identify the most important nucleotide locations from a diagnostic perspective without significantly harming the accuracy in identifying mutations that are indicative of the given cancer type.

In some implementations, the computing system can include and/or utilize a mutation analysis mechanism that identifies a set of unique portions or segments in the human genome/DNA and related mutations that correspond to development/onset of certain types of cancer. The computing system can identify the set of unique portions or phrases and mutations (e.g., text strings having a length of k) based on the TRs.

Second, the efficiencies of the improved diagnoses and/or prediction system can be further leveraged to protect the sensitive data by managing errors or noises generated during processing of encrypted data, such as for reducing/eliminating noise propagation during artificial intelligence (AI)-based analysis of encrypted data. As an illustrative example, the computing system can use homomorphic encryption (HE) to encrypt the training data, the ML model, the patient health data to be provided to the ML model as input, the output produced by the ML model, or a combination thereof.

HE may include an encryption mechanism that allows devices/systems to perform computations on its encrypted form (also referred to as “ciphertext” or CT) without accessing the decrypted form of the data (also referred to as “plaintext” or PT). HE can provide such operability by representing the computations as Boolean or arithmetic circuits, such as using different amounts, types, and/or layering/depth of logical gates (e.g., AND, OR, NOT, etc.). Some examples of HE include lattice-based cryptography, Benaloh cryptosystem, multivariate quadratic full HE (FHE), Paillier encryption scheme, ElGamal encryption scheme, Matrix Operation for Randomization or Encryption (MORE) scheme, and the like.

In leveraging the operability of HE, a separate party/entity (e.g., a patient or healthcare provider) may provide the CT data while withholding the encryption key. The computing system can be configured to process the CT (e.g., by generating the ML models and/or the output) without decrypting and accessing the PT. The corresponding ML models and the output can remain encrypted so that no other users can access the PT data, thereby protecting the privacy of the patient.

Such benefits of HE may be realized by overcoming limitations or issues unique or characteristic of HE. For example, HE schemes may generate and propagate errors (also referred to as “noise”) during the CT computations. When unmanaged, the noise grows with the increasing number of operations and may ultimately corrupt the output result into an indecipherable form or an incorrect PT value.

The computing system can be configured to manage the HE-based noise during CT computations. For example, the computing system can implement during the CT computations a bootstrapping mechanism (e.g., application of decrypting procedures or re-encryption based on encrypting the CT anew) that refresh the CT and reduce or eliminate the existing noise. In other words, the computing system can implement the bootstrapping mechanism at key points during the computations to reduce or eliminate the noise.

The computing system can implement the bootstrapping mechanism on the intermediate processing results using an encoded/CT version of a key, such as a public key or an evaluation key related to the secret key used to encode the data or an encoded version of the secret key, associated with the encryption and a decryption mechanism (e.g., a circuit representation of the decryption scheme). For example, the computing system can encrypt the CT data (e.g., the intermediate data) to generate doubly encrypted data. The computing system can remove the inner encryption by homomorphically evaluating the doubly encrypted data and the encrypted decryption key using the decryption mechanism. As a result, the computing system can generate the encrypted output (i.e., without accessing the PT content) that is refreshed and having reduced noise.

For illustrative purposes, noise mitigation is described as using the bootstrapping mechanism. However, it is understood that the computing system can additionally or alternatively use other mechanisms or subsystems, such as filters, Residue Number System (RNS), Learning with Errors (LWE) system, Brakerski-Gentry-Vaikuntanathan (BGV) system, or the like.

In some implementations, the computing system can be configured to implement the noise mitigation in segments and/or at randomly-selected points in the computation to further enhance data security. Using neural networks as an illustrative example, the computing system can implement the bootstrapping mechanism at randomly selected neural nodes. For the random selection, the computing system can calculate a maximum number of computations that correspond to allowable noise propagation based on one or more aspects of the input data (e.g., bit length, encryption strength, encryption error-correction capacity, number of targeted phrases or TRSs, and/or number of input/output parameters). The computing system can use the maximum number of computations to determine a corresponding threshold quantity of nodes and/or layers. The computing system can use the threshold node/layer quantity as a boundary for implementing the noise mitigation mechanism. In other words, the computing system can be configured to implement/complete the boot strapping mechanism within the node/layer boundary.

Within the boundary, the computing system can randomly or pseudo-randomly select points or timings (e.g., nodes) to implement the noise mitigation mechanism during model generation and/or implementation. The selected points can correspond to noise mitigation for a portion within the overall data. Accordingly, the computing system can use a select mechanism (e.g., linear feedback shift registers (LFSR) based on prime polynomials) configured to generate a set of selections that combine to mitigate the noise for the overall data. For example, the computing system can randomly select an initial set of one or more neural nodes to implement the bootstrapping mechanism. Based on the coverage of the initial set, the computing system can determine one or more subsequent/complementary sets of nodes required to cover the remaining portions of the processed data. Thus, the computing system can provide enhanced privacy protection by randomly implementing the noise mitigation and/or by implementing the mitigation within an encrypted model while preserving the integrity, accuracy, and decipherability of the processed data.

In some implementations, the ML model can be configured to divide the input data into segments and combine the segmented results. The computing system can (randomly) select the bootstrapping points within the segments and/or separately during the combining process. By segmenting the data, the computing system can reduce the computational load required to select the mitigation points that fully cover the processed data. Further, the computing system can use the segmenting process to further introduce privacy protection, such as by utilizing different sizes and/or rules for the segments.

Within the trained model and/or during generation/training of the model, the computing system can input the ciphertext into a selected node, perform the node function, and then implement the bootstrapping mechanism (e.g., decryption/encryption described above). Alternatively or additionally, the computing system can input the ciphertext into the selected node, implement the bootstrapping mechanism, and then perform the node function. In some implementations, the computing system can input the ciphertext into the selected node, implement a portion of the noise mitigation mechanism (by, e.g., decrypting the data), perform the node function, and then implement another/remaining portion of the noise mitigation mechanism (by, e.g., encrypting the data). The computing system may be further configured to use and randomly select from different sequence combinations at the selected node.

In addition to the randomly implementing the noise mitigation, the computing system can further enhance the data protection by using dummy nodes or operations and/or dedicated noise mitigation nodes. The computing system can use the dummy nodes/operations to create false paths and/or false functions that are removed during subsequent computations or do not contribute to the overall outcome. The computing system can use dedicated mitigation nodes to only implement the noise mitigation (e.g., without other node function) to protect against any nefarious attempts to reverse engineer the model.

For illustrative purposes, various implementations of the technology are described in the context of developing ML models. In the development stage, ML models can be described as stochastic or deterministic. The terms “stochastic” and “non-deterministic” refer to algorithms that, even for the same training data, can exhibit different behaviors on different runs through the computational architecture. Examples of stochastic ML models include those based on neural networks, random forests, gradient descent algorithms, and the like. Note that while these ML models may be considered stochastic during development, these ML models are generally considered deterministic once the internal workings (e.g., weights) are learned. Meanwhile, the term “deterministic” refers to algorithms that, given a certain input, will always produce the same output and always pass through the computational architecture in the same way.

Accordingly, the approach to mitigating noise that is mitigation described herein can be applied to various ML models, including those based on support vector machines (SVMs), logistic regression, random forest, residual learning, active learning, generative adversarial networks (GANs), etc.

For brevity, implementations of the technology are described in the context of detecting cancer using genetic information (e.g., deoxyribonucleic acid (DNA) sequence listings) or other patient-specific data. However, it is understood that the described technology may be used to process sensitive or protected information in other applications or contexts, such as in detecting the presence of other diseases or disease conditions, stratifying a patient among different disease conditions for treatment purposes, processing protected test/product data, processing financial transactions or banking information, cloud computing, blockchain, and the like.

Implementations may be described in the context of instructions that are executable by a system for the purpose of illustration. However, those skilled in the art will recognize that aspects of the technology described herein could be implemented via hardware, firmware, or software. As an example, a computer program that is representative of a software-implemented genetic information processing platform (or simply “processing platform”) designed to process genetic information may be executed by the processor of a system. This computer program may interface, directly or indirectly, with hardware, firmware, or other software implemented on the system. Moreover, this computer program may interface, directly or indirectly, with computing devices that are communicatively connected to the system. One example of a computing device is a network-accessible storage medium that is managed by a healthcare entity (e.g., a hospital system or diagnostic testing facility).

### Overview of Genetic Information Processing System

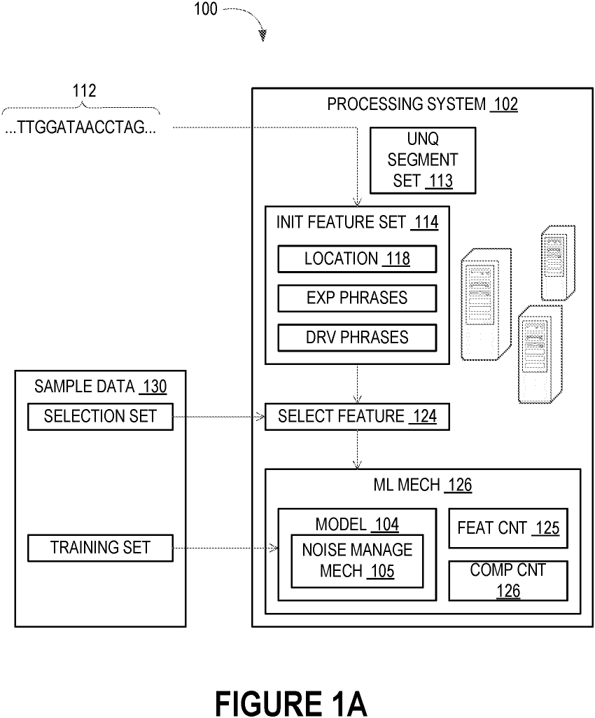

FIGS. 1A and 1B show example operating environments of a computing system 100 including a genetic information processing system 102 (“processing system 102”) in accordance with one or more implementations of the present technology. The processing system 102 can include one or more computing devices, such as servers, personal devices, enterprise computing systems, distributed computing systems, cloud computing systems, or the like. The processing system 102 can be configured to analyze DNA information for diagnosing one or more types of cancer, for evaluating development stages leading up to the onset of the one or more types of cancer, and/or for predicting a likely onset of the one or more types of cancer.

The application environment depicted in FIG. 1A can represent a development or training environment in which the processing system 102 develops and trains an analysis mechanism, such as a ML model 104, configured to detect a presence, a progress, and/or a likely onset of one or more types of cancer. The ML model 104 can include a sequence of functions or operations (e.g., mathematical, logical, comparisons, conditionals, or the like) along with corresponding trained factors that produce an output value representative of the input value. In developing and training the ML model 104, the processing system 102 can first identify an analysis template (e.g., specific data locations or values within a reference data 112, such as the human genome or other data derived from human/patient DNA) targeted for further analysis/consideration.

As an illustrative example, the processing system 102 can use a text-based representation (e.g., one or more text strings) of the human DNA as the reference data 112. The processing system 102 can analyze the reference data 112 to identify specific locations and/or corresponding text sequences that can be utilized as identifiers or comparison points in subsequent processing. In some implementations, the processing system 102 can use a set of unique text segments 113 (e.g., a set of unique TRs) found or expected in the reference data 112 to generate an initial feature set 114. The processing system 102 can generate the initial feature set 114 by identifying expected phrases that include the unique segment set 113 and/or by computing derivations thereof (e.g., derived phrases) that represent mutations targeted for analysis. The initial feature set 114 and/or the unique segment set 113 can include location identifiers 118 associated with a relative location of such segments, phrases, and/or derivations within the reference data 112.

For the feature selection, the processing system 102 can iteratively add or remove one or more unique locations/sequences and/or derivations from the initial feature set 114 and calculate a correlation or an effect of the removed data point on duplicating the known classifications of sample data 130 (e.g., DNA information that is (1) known to be characteristic of corresponding types of data or (2) collected from patients confirmed to have the corresponding types of data), such as to accurately recognize the different categories of the sample data 130. The processing system 102 can determine a set of selected features 124 that correspond to the unique locations/phrases and derivations thereof having at least a threshold amount of affect or correlation with one or more corresponding cancer types. In other words, the processing system 102 can determine the set of features 124 including locations, sequences, specific mutations, or combinations thereof that are deterministic/characteristic of or commonly occurring in corresponding cancers. Based on the selected set of features 124, the processing system 102 can implement a ML mechanism 126 (e.g., random forest, neural network, etc.) to generate the ML model 104. The processing system 102 can further train the ML model 104 using training data.

Using the set of features (e.g., targeted text segments/phrases based on the unique TRs), the processing system 102 can limit the amount of data considered or processed in subsequent analyses, such as in feature selection, model generation, model training, and/or the like. For example, the processing system 102 can use the targeted segments/phrases to reduce the size of analyzed data. Accordingly, the processing system 102 can reduce the resource consumption through the decreased data size of the selected feature set.

In some implementations, the ML mechanism 126 can be configured to process encoded (e.g., CT) data. For example, the ML mechanism 126 can be configured to identify and use PT operations that preserve the intended CT manipulations. Additionally or alternatively, the ML mechanism 126 can use CT operations to provide the intended operations. Accordingly, the ML mechanism 126 can use the sample data 130 that includes PT-only, CT-only, or both to train and develop the model 104. Additionally or alternatively, the ML mechanism 126 can train an initial PT model using PT sample data, and then encode the resulting model or a portion thereof, such as according to HE mechanisms. The resulting model 104 can be configured to analyze the CT data and generate the corresponding cancer signature (e.g., a determination that the patient providing the DNA information has or is likely to develop a certain type of cancer or a corresponding score) without decrypting and accessing the PT version of the input data.

To maintain accuracy of the model 104, the ML mechanism 126 can include a noise management mechanism 105, such as instructions, operations, corresponding circuit components or models, or the like that selectively mitigate or reduce noise generated during operation of the model 104. As described above, the model 104 can introduce or generate unintended noise or errors over multiple operations while processing HE CT data. The model 104 can be configured to initiate the noise management mechanism 105 (e.g., bootstrapping operation) selectively and/or periodically to reduce or remove the noise from intermediate processing results. Accordingly, the model 104 can stop or reduce propagation of the noise and preserve the accuracy of the derivation results.

The model 104 can be configured to trigger the noise management mechanism 105 based on one or more aspects of the expected input data. For example, the trigger timing can be derived either statically or dynamically based on the input data format (e.g., a sequence of counts that each refer to a specific phrase, mutative or otherwise, at a targeted location in the genome) and/or the data size of the received patient input. Additionally or alternatively, the trigger timing can be derived according to the expected computations of the model 104. For example, the trigger timing can be associated with the worst-case computations, potential computational paths/sequences, or the like. Accordingly, the ML mechanism 126 can use a feature count 125 (e.g., a total number of phrases) of the selected features to derive or configure the trigger timing. Also, the ML mechanism 126 can configure the model 104 to track a computation count 126 during operation thereof (e.g., for each patient DNA input). The model 104 can be configured to trigger the noise management mechanism 105 when the computation count 126 satisfies a threshold.

The application environment depicted in FIG. 1B can represent a deployment environment in which the processing system 102 applies the analysis mechanism to detect a presence, a progress, and/or a likely onset of one or more types of cancer from patient DNA data. As an illustrative example, the computing system 100 can include a sourcing device 152 that provides an input 132 and/or receives a result 134 representative of the presence, the progress, and/or the likely onset of the one or more types of cancer for the input 132.

The sourcing device 152 can include any of a variety or type of computing devices, such as a notebook or laptop computer, a multimedia computer, a desktop computer, a grid-computing resource, a virtualized computer resource, a cloud computing resource, a sequencing device, or a combination thereof. The sourcing device 152 can be operated by a patient submitting the evaluation target 132, a healthcare service provider associated with the patient, an insurance company, or the like. Accordingly, the sourcing device 152 can be configured to receive a PT input 166 that includes one or more sequences of plaintext letters representative of the patient DNA information or related information. In other words, the PT input 166 can include DNA sequencing result or a further derived result (e.g., counts of targeted phrases found therein) from a patient sample.

In some implementations, the computing system 100 can include a sourcing module 162 operating on the source device 152. The sourcing module 162 can include a device/circuit and/or a software module (e.g., a codec, an app, or the like) that generates or pre-processes the evaluation target 132. For example, the sourcing module 162 can include a homomorphic encoder that encrypts and prevents unauthorized access to the patient data. The evaluation target 132 can include the homomorphically encoded CT data that can be processed at the processing system 102 without fully decrypting and recovering the patient PT data. In other words, the processing system 102 can apply the ML model 104 that is configured to process or perform computations on the encrypted data.

The sourcing module 162 can process and encode the PT input 166, such as using one or more keys 164 to generate the encoded CT input 132 (also referred to as the evaluation target 132). The sourcing device 152 can provide the evaluation target 132 (e.g., the CT input) to the processing system 102 but maintain at least one of the keys 164 private and unrevealed to the processing system 102. In other implementations, the sourcing device 152 can provide an encrypted form of the key 164 to the processing system 102. In other words, the CT data 132 may include the encrypted forms of both the PT input 166 and the key 164. The sourcing device 152 may provide the ciphertext data of the encryption key using a chain of public-private key pairs, where the private key (also referred to as the “secret key”) is encrypted under the next public key.

The processing system 102 can receive and test the CT evaluation target 132 against the model 104. Accordingly, the processing system 102 can generate an evaluation result 134 that represents a cancer diagnosis or a cancer signal. For example, the evaluation result 134 can represent a determination that the patient has cancer, a stage (e.g., clinically recognized stages 1-4) of the onset cancer, a progress state before/leading up to an onset state of cancer, a likelihood of developing cancer within a predetermined period, an identification of the type of cancer, or a combination thereof.

Based on the encryption, the processing system 102 can process the CT evaluation target 132 as discussed above without accessing the PT input 166. From the input to the final output, the processing system 102 can process the information in the encrypted format, such as by leveraging the HE properties. The processing device 102 can maintain the encrypted format across intermediate processing results and the final result data. The generated evaluation result 134 can be CT and thus remain private or encoded to the processing system 102 or any users thereof.

Operating on or computing the encrypted form of data can introduce noise (e.g., potential changes or errors) in the processed data. In other words, the intermediate results may include errors that may be further amplified by subsequent computations.

The encryption mechanism may have some built-in error correction capability for eliminating noise and accurately deciphering the encoded data. However, when noise persists, propagates, or otherwise grows beyond a threshold level, the resulting errors or corruption in data can exceed the error correction capacity, thereby preventing accurate recovery of the content. In other words, noise exceeding threshold levels can overwhelm and corrupt the processed data and negatively affect the deciphering process. The propagation of noise and the potential to corrupt data may be amplified in analyses that require relatively large numbers of computations, such as for artificial intelligence-based analysis and/or for complex encryptions (e.g., HE).

The processing system 102 and/or the model 104 can include the noise management mechanism 105 configured to manage such noise across the data computations as described above. The noise management mechanism 105 can include software, hardware, firmware, or a combination thereof that is configured to reduce or eliminate noise created during or by computing/operating on encrypted ciphertext data. For example, the noise management mechanism 105 may be designed, programmed, or trained to selectively implement or execute bootstrapping functions to “refresh” HE CT data during application of the ML model 104. In other implementations, the noise management mechanism 105 can include a filter, an error correction routine, or the like. Also, the noise management mechanism 105 can manage the noise based on reducing the modulus of the ciphertext space along with the noise.

To further enhance the security of the processed data, the processing system 102 can selectively/randomly implement the noise management mechanism 105 across the computations. In some implementations, the processing system 102 can use a threshold boundary (e.g., a maximum number of allowable computations or a minimum triggering threshold) in addition to one or more characteristics of the input data, the computation count 126 of FIG. 1A, or the like. The threshold boundary can correspond to a tolerable level of noise where the content remains decipherable. The processing system 102 can select different computational points within the threshold boundary during the overall analysis. The processing system 102 can select the computational points (e.g., neural nodes, decision points, functions, etc.) randomly or pseudo-randomly, such as using LFSR with prime polynomials. The processing system 102 can select the computational points according to predetermined rules or processes that refresh all or select portions of the processed data. Details regarding the noise management mechanism 105 and the implementation thereof are described below.

The processing system 102 can include a pre-processing module 164 that conditions the evaluation target 132 for the model application. For example, the pre-processing module 164 can include circuits and/or software instructions that are configured to remove biases or noises introduced before receiving the evaluation target 132.

### Data Processing Formats

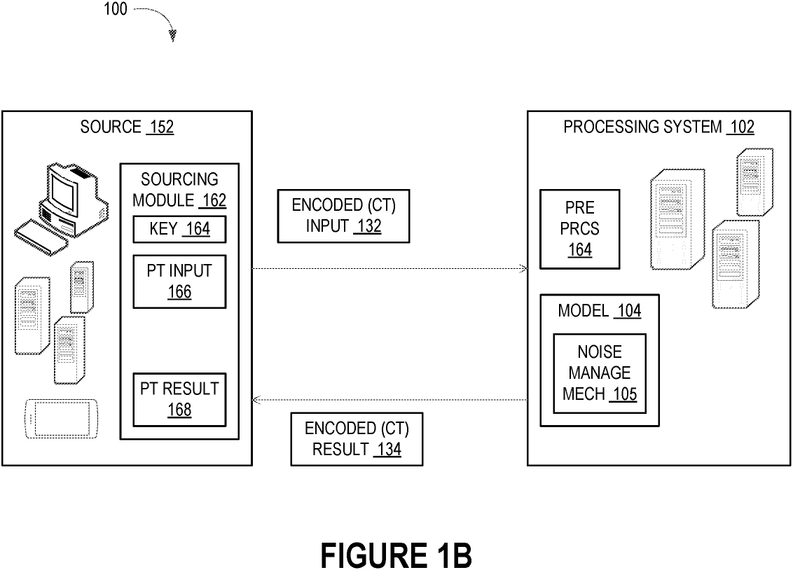

In developing/training the model 104 and/or deploying the model 104, the computing system 100 can utilize one or more data processing formats (e.g., data structures, organizations, inputs/outputs, or the like). FIG. 2 shows an example data processing formats for the processing system 102 in accordance with one or more implementations of the present technology. The processing system 102 can receive and process a DNA sample set 206 (e.g., an instance of the reference data 112 and/or sample data 130 illustrated in FIG. 1A) having one or more of the formats or subfields illustrated in FIG. 2. Moreover, the processing system 102 can generate the initial feature set 114 (FIG. 1A) using one or more detailed example aspects depicted in FIG. 2.

As an illustrative example, the DNA sample set 206 can include DNA data (e.g., representative of a set of sequenced DNA information) corresponding to different known categories. Examples of the DNA sample set 206 can include genetic information (e.g., text-based representations) derived or extracted from human bodies, such as from tissue extracted during a biopsy or from cell-free DNA (e.g., DNA that is not encapsulated within a cell) in bodily fluids. The DNA sample set 206 can include DNA data collected from volunteers or participating patients having medically confirmed diagnoses and/or from public or private databases.

The DNA sample set 206 can include data collected from different types/categories of samples, such as cancer-free samples (cancer-free data 210), non-cancerous regions/samples (non-regional data 211), and/or cancerous samples (cancer-specific data 212). The cancer-free data 210 can represent text-based DNA data corresponding to samples collected from patients confirmed/diagnosed to be cancer free. The non-reginal data 211 can represent text-based DNA data corresponding to samples collected from non-cancerous regions (e.g., white blood cells or leukocytes) of patients confirmed/diagnosed to have one or more types of cancer. The cancer-specific data 212 can represent text-based DNA data corresponding to samples (e.g., tumor biopsies, liquid biopsies, etc.) collected from cancerous regions or tumors confirmed/diagnosed to be a specified type of cancer. The DNA sample set 206 can include information (e.g., the non-regional data 211 and/or the cancer-specific data 212) corresponding to one or more types of cancers (e.g., breast cancer, lung cancer, colon cancer, and/or the like).

The DNA sample set 206 can further include descriptions regarding a strength or a trustworthiness of the data. For example, the DNA sample set 206 can include a sample read depth 214 and/or a sample quality score 216. The sample read depth 214 can represent a number of times a given nucleotide in the genome (e.g., certain text string/portion) was detected in a sample. The sample read depth 214 may correspond to a sequencing depth associated with processing fragmented sections of the genome within a tissue sample. The sample quality score 216 can represent a quality of identification of the nucleobases generated by DNA sequencing. In some implementations, the sample quality score 216 can include a phred quality score.

The DNA sample set 206 can also include supplemental information 220 that describes other aspects of the sample or the source of the data. For example, the supplemental information 220 can include information such as sample specification information 222 (or simply “specification information”), sample source information 224 (or simply “source information”), patient demographic information 226, or a combination thereof.

The specification information 222 can include technical information or specifications about the sequenced DNA associated with the DNA sample set 206. For example, the specification information 222 can include information about the locations 118 (FIG. 1A) within the genome to which the DNA fragments correspond, such as intron and exon regions, specific genes, or chromosomes. Also, the specification information 222 can describe, e.g., (1) the process, methods, and instrumentation used to extract and sequence the genetic material, (2) the number of sequencing reads for each sample, or a combination thereof.

The source information 224 can include details regarding the source and/or the categorization of the sample. For example, the source information 224 can include information about the cancer type, the stage of cancer development, the organ or tissue from which the sample was extracted, or a combination thereof.

The patient demographic information 226 can include demographic details of the patient from which the sample was taken. For example, the patient demographic information 226 can include the age, the gender, the ethnicity, the geographic location of where the patient resides/visited, the duration of residence/visitation, predispositions for genetic disorders or cancer development, family history, or a combination thereof.

The processing system 102 can analyze the DNA sample set 206 using the mutation analysis mechanism. Accordingly, the processing system 102 can identify mutations or mutation patterns in specific DNA sequences that can be used as markers to determine the existence, the progress, and/or the developing stages of a particular form of cancer. To identify the relevant mutations, the processing system 102 can detect a set of targeted locations or text patterns (according to, e.g., the TRs) within the reference genomes.

The processing system 102 can generate and/or utilize a genome tandem repeat reference catalogue 230 that represents a catalogue or a collection of uniquely identifiable TRs in the human genome. As an example, the genome tandem repeat reference catalogue 230 can be based on a reference human genome (e.g., the reference data 112), such as the GRCh38 reference genome. The uniquely identifiable sequences can include DNA sequences having therein a series of multiple instances of directly adjacent identical repeating nucleotide units or base patterns, such as microsatellite DNA sequences. The base patterns can have a predetermined length, such as one for a repetition of one letter or monomer (e.g., ‘AAAA’) or greater (e.g., three for tetramers, such as ‘ACT’). Such uniquely identifiable TRs can serve as reference sequences (e.g., reference locations within the human genome) or markers for evaluating the DNA sample set 206. Since the DNA sample set 206 may correspond to incomplete DNA fragments, the unique TRs found within the fragments may be used to map the DNA information to the human genome.

The processing system 102 can use the genome tandem repeat reference catalogue 230 to compute the initial feature set 114. For example, the processing system 102 can use the unique TRs identified in the genome tandem repeat reference catalogue 230 to generate derived strings that represent potential mutations. In some implementations, the processing system 102 can identify text characters preceding and/or following each unique TR and derive the mutation strings that represent one or more types of mutations (e.g., insert-deletion (indel) mutations). Details regarding the initial feature set 114 (e.g., strings with flanking characters and/or mutation strings) are described below.

The processing system 102 can compare the mutations at the targeted locations/patterns across the different types of DNA sample set 206. Based on the comparison, the processing system 102 can compute a correlation between or a likely contribution of the mutations at the targeted locations/sequences and the development of cancer. Accordingly, the processing system 102 may generate a cancer correlation matrix 242 that correlates identified tumorous sequences or text-based patterns to specific types of cancer. For example, the cancer correlation matrix 242 can be an index that includes multiple instances of the uniquely identifiable tandem repeat sequences in the genome TR reference catalogue 230 that, when found to be tumorous, indicate the existence of a particular form of cancer or indicate the possibility that a particular form of cancer will develop.

The processing system 102 can perform the feature selection using the cancer correlation matrix 242, such as by retaining the locations/patterns and/or derived mutation patterns having at least a predetermined degree of correlation to one or more corresponding types of cancer. Using the selected features, the processing system 102 can develop and train the ML model 104 configured to detect, predict, and/or evaluate development or onset of cancer.

### Base Text Patterns - Expected Phrases

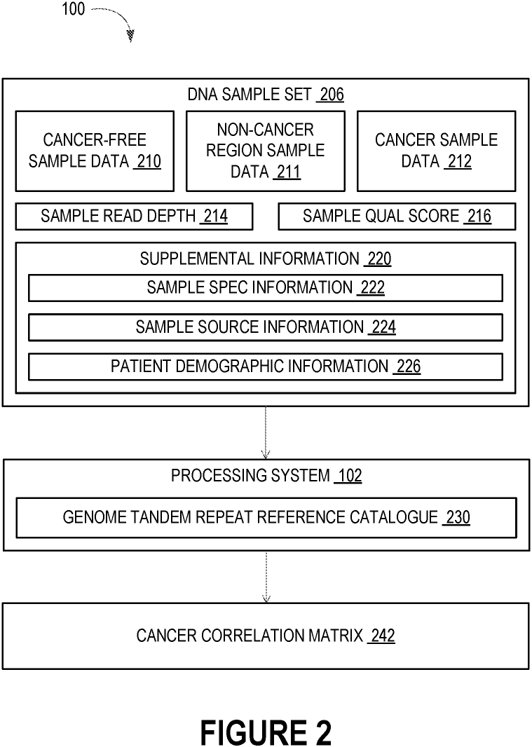

The processing system 102 can use segments (e.g., the unique segment set 113) to generate phrases. FIG. 3 shows example expected phrases 310 in accordance with one or more implementations of the present technology. The expected phrases 310 can correspond to textual representations of the DNA sequences or a set of sequence variations that may be used as bases for subsequent processing/comparisons, such as in deriving mutations strings and analyzing the DNA sample set 206 (FIG. 2).

For context, samples collected from patients may include fragments or portions of the overall DNA. As such, the corresponding sequenced values or the text string may include different combinations of characters. The processing system 102 (FIG. 1A) can generate the expected phrases 310 as representations of different character combinations that include the uniquely identifiable segments (e.g., the unique segment set 113 3 3). 3 3 3 3 In some implementations, the processing system 102 can generate a set (illustrated as a unique sequence identifier number in FIG. 3) of the expected phrases 310 for each unique segment 360 (illustrated using bolded characters in FIG. 3).

The expected phrases 310 can have a phrase length 316 of k (e.g., between 10 to 50 or more) number of DNA base pairs or pairs of nucleobases. Each DNA base pair can be represented as a single text character (e.g., ‘A’ for adenine, ‘C’ for cytosine, ‘G’ guanine, and ‘T’ thymine). As such, the expected phrases 310 may also be referred to as “k-mers.”

In some implementations, as described above, the unique segment 360 can include a DNA sequence, of a specified minimum length. The unique segment 360 can include a series of multiple instances of directly adjacent identical repeating nucleotide units or repeated base units 356. For example, the unique segment 360 can include a minisatellite DNA or microsatellite DNA sequence of a specified minimum length. Accordingly, the unique segment 360 can correspond to a repeated pattern of the repeated base units 356, and the number of repetitions can correspond to a segment length 320 (e.g., the total length of, or total number of, nucleotide base pairs) for the unique segment 360. The repeated base unit 356 can have a base unit length 324 corresponding to the number of nucleotides within the repeated base unit 356 (e.g., one for a mononucleotide, two for a di-nucleotide, etc.).

For illustrative purposes, FIG. 3 shows a specific instance for the unique segment 360 of “AAAAAAAA,” annotated as “A8,” located at the molecular position starting at “10,513,372” on chromosome 22. In this example, the unique segment 360 includes the segment length 320 of eight base pairs with the repeated base unit 356 of one base pair (e.g., a monomer or a mononucleotide) ‘A.’

The processing system 102 can use the phrase length 316 (e.g., k between 10 to 50 or more base pairs) that has been predetermined or selected to capture targeted amount of data/characters surrounding the unique segments 460. As such, the phrase length 316 can be greater than the segment length 320, and each of the expected phrases 310 can include a set of flanking texts 314 (e.g., text-based patterns; illustrated using italics in FIG. 3) preceding and/or following the corresponding unique segment 360.

The processing system 102 can generate the expected phrases 310 in a variety of ways. As an illustrative example, the processing system 102 can use each of the unique segments 360 as an anchor for a sliding window having a length matching the phrase length 316. The processing system 102 can iteratively move the sliding window relative to the unique segment 360 and log the text captured within the window as an instance of the expected phrases 310. As such, each of the expected phrases 310 can correspond to a unique position of the sliding window relative to the unique segment 360. Also, the set of expected phrases 310 for one reference TR can include different combinations of the flanking text 314 (e.g., a combination of one or more leading characters 332 and/or one or more tailing characters 334).

The total number of base pairs in flanking text 314 can be a fixed value that is based on the phrase length 316 and the segment length 320. The number of characters in the flanking text can be calculated as the difference between the phrase length 316 and the segment length 320. As an example, for one of phrases having a length of 21 base pairs and a segment length of 8 base pairs, the flanking text can include13 base pairs/characters.

Each of the expected phrases 310 can represent one of a number of position variant k-mers based on the flanking texts 314. The position variant k-mers can include specific numbers of base pairs in the expected flanking text 332 and tailing flanking text 334. For example, a set of the expected phrases 310 can include the same unique segment (e.g., repeated pattern of the TR) and differ from one another according to the number of base pairs included in the leading flanking text 332 and/or the tailing flanking text 334. In general, the number of base pairs included in the leading flanking text 332 and tailing flanking text 334 can vary inversely between the different instances of the position variant k-mers or expected phrases 310.

As an example, each of the expected phrases 310 illustrated in FIG. 3 has the phrase length 316 of 21 base pairs and the segment length 320 of 8 base pairs. A first expected phrase can have the leading characters 332 corresponding to 12 base pairs and the tailing character 334 corresponding to 1 base pair. A second expected phrase can have the leading characters 332 corresponding to 11 base pairs and the tailing characters 334 of 2 base pairs. The pattern can be repeated until the last expected phrase has the leading characters 332 corresponding to 1 base pair and the tailing characters 334 corresponding to 12 base pairs.

The expected phrases 310 can be grouped into sets that each correspond to a unique segment as described above. The total number of phrases or position variant k-mers (position variant total) in the grouped set can be represented as:

$$\text{Position Variant Total =} \left(\text{Phrase length} k\right) \text{-} \left(\text{Segment length}\right) \text{- 1} \text{.}$$

For the example illustrated in FIG. 3, the set of expected phrases can have a position variant total of 12, representing 12 different instances of phrases corresponding to the phrase length 316 of 21 and the segment length 320 of 8.

In some implementations, the processing system 102 can use the unique instances of the TRs as the basis for generating the sets of expected phrases 310. Accordingly, each of the expected phrases 310 can also be unique since it is generated using the corresponding unique TR as a basis. The processing system 102 can use the unique expected phrases 310 to account for and identify the fragmentations likely to be included in the patient samples.

### Base Text Patterns - Derived Phrases

The processing system 102 can use the expected phrases to analyze mutations in genetic information (e.g., sequenced DNA segments), such as for detecting tumorous/cancerous DNA sequences. The expected phrases can be used to detect locations within the reference genome and related mutations that are indicative of certain types of cancers or likely onset thereof. The processing system 102 can use the expected phrases as basis to generate derived phrases that represent various mutations in the genetic information. The processing system 102 can use the derived phrases to recognize or detect mutations in the DNA sample set 206 (FIG. 2), the sample data 130 (FIG. 1A), or the like in developing, training, and/or deploying the ML model 104. Effectively, the processing system 102 can identify the mutation patterns indicative of certain types of cancers based on using the derived phrases to determine differences between healthy and cancerous DNA samples (between, e.g., the cancer-free data 210, the non-regional data 211, and/or the cancer-specific data 212 illustrated in FIG. 2).

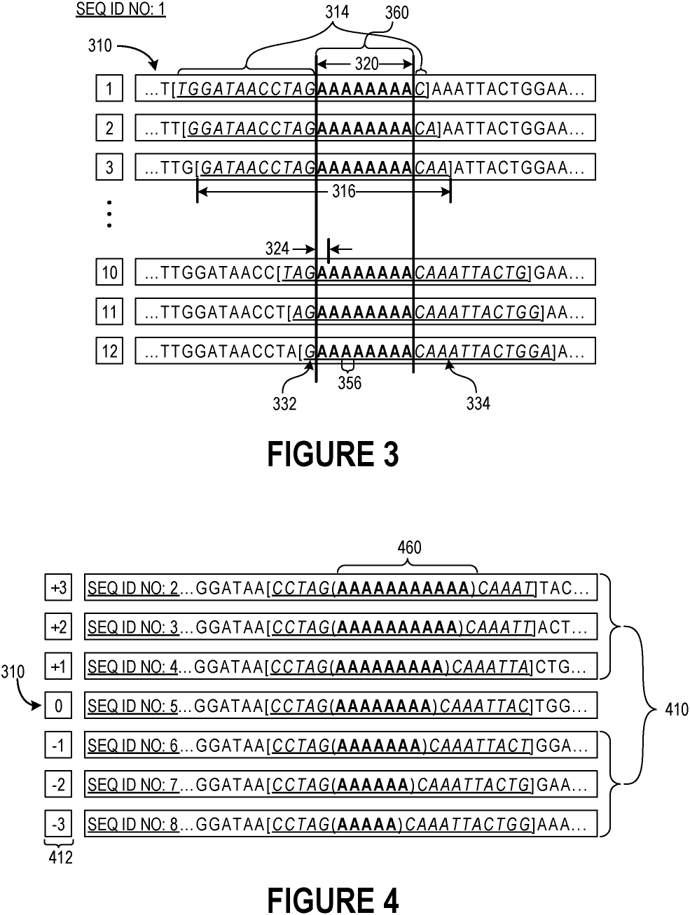

FIG. 4 shows example derived phrases 410 in accordance with one or more implementations of the present technology. The processing system 102 (FIG. 1A) can generate the derived phrases 410 based on adjusting the expected phrases 310 expected to a predetermined pattern. For example, for one or more or each expected phrase 310, the processing system 102 can generate a set of the derived phrases 410 that represent indel mutations of the corresponding expected phrase 310. In some implementations, the processing system 102 can generate the set of derived phrases 410 that correspond to a predetermined number of insertions and/or deletions in the unique segment 360 (FIG. 3) within the corresponding expected phrase 310. In other words, the set of derived phrases 410 can represent the indel variants of the sequence represented by the corresponding expected phrase 310.

The processing system 102 can generate the set of the derived phrases 410 based on adjusting (via insertion/deletion) the number of the repeated base units 356 (FIG. 3) and/or one or more characters in the unique segment 360 of the expected phrase 310. Accordingly, the processing system 102 can generate a set of derived segments 460 that correspond to indel variants of the unique segment 360.

The processing system 102 can generate the derived phrases 410 based on adding and/or adjusting the flanking text 314 (FIG. 3) around the derived segments 460 (illustrated as the bolded characters within parentheses ‘()’). In some implementations, the processing system 102 can generate the derived phrases 410 having the same phrase length 316 (FIG. 3) as the expected phrases 310. As a result, the processing system 102 can expand or reduce the coverage of the flanking text 314 according to the indel changes to the unique segment 360 (e.g., the originating pattern of TRs). With deletions, the processing system 102 can include corresponding number of new characters from the overall sequence into the flanking text 314 (FIG. 3). Similarly with additions, the processing system 102 can remove the corresponding number of characters from the flanking text 314. For illustrative purposes, FIG. 4 shows the surrounding adjustments occurring in the trailing characters 334 (FIG. 3) while maintaining the leading characters 332 (FIG. 3). However, it is understood that the processing system 102 can operate differently, such as by (1) adjusting the leading characters 332 while maintaining the trailing characters 334 and/or (2) spreading the adjustments across the leading characters 332 and the trailing characters 334 according to the number of characters in the original phrase and/or a predetermined pattern.

For the example illustrated in FIG. 4, the expected phrase 310 can correspond to the repeated TR segment of “AAAAAAAA” or A8 beginning at position 10,513,372 on chromosome 22. The derived phrases 410 can correspond to the derived segments 460 including up to three insertions and deletions of the repeated base unit ‘A.’ In other words, the derived phrases 410 can correspond to phrases built around A5, A6, A7, A9, A10, and A11.

The number of the derived phrases 410 associated with a given expected phrase can be determined by an indel variant value 412. The indel variant value 412 can include an integer value representative of the number of insertions and deletions. The indel variant value 412 can further function as an identifier for a phrase. For example, the indel variant value ‘0’ can represent the expected phrase 310 having zero insertions/deletions. Positive indel variant values (e.g., 1, 2, 3) can represent derived phrases including corresponding number of insertions of base units or characters in the repeated TR portion. Negative indel variant values (e.g., -1, -2, -3) can represent derived phrases corresponding number of deletions of base units or characters in the repeated TR portion. For the example illustrated in FIG. 4, the indel variant values 1, 2, and 3 can represent/identify A9, A10, and A11, respectively. Also, the indel variant values -1, -2, and -3 can represent A7, A6, and A5, respectively.

For context, the processing system 102 can use the expected phrases 310 and the corresponding sets of derived phrases 410 to analyze the DNA sample set 206 and develop/test the ML model 104 (FIG. 1A). The phrases generated using the unique TR patterns can provide accurate and precise identification of corresponding sequences in the different types of health and cancerous DNA samples. In other words, the various phrases can represent the type of textual patterns or the corresponding sequences that are targeted for analyses and comparisons between the cancer-free data 210, the non-regional data 211, and/or the cancer-specific data 212. For example, the processing system 102 can use the various phrases to identify the numbers and types/locations of mutations in the cancer-related samples and absent in healthy samples. The processing system 102 can aggregate the results across multiple samples and patients to derive a pattern or a correlation between certain types of mutations and the onset of certain types of cancer.

To put things another way, the processing system 102 can identify unique patterns (e.g., the unique TR patterns and/or the corresponding expected phrases 310) that each occur once within the human genome. The unique patterns can be used to identify specific locations and portions within the human genome for various analyses. Moreover, the processing system 102 can target specific types of mutations, such as indel mutations, in developing a cancer-screening and/or a cancer-predicting tool. It has been found that various types of cancers can be accurately detected and progress/status of such types of cancers can be described using the expected phrases 310 and the corresponding sets of the derived phrases 410 (e.g., sequences identified using unique TR-based patterns and indel variants thereof) and without considering other aspects/mutations of the human DNA. As a result, the processing system 102 can generate the ML model 104 that can accurately detect the existence, predict a likely onset, and/or describe a progress of certain types of cancers using the various phrases. In other words, the processing system 102 can detect/predict the onset of cancer without processing the entire DNA sequence and different types of mutation patterns.

The processing system 102 can further improve the efficiency and reduce the resource consumption using the indel variant value 412. Given the downstream processing methodology, the indel variant value 412 can control the number of phrases considered in developing/training the ML model 104 and thereby affect the overall number of computations and the amount of resource consumption. When the indel variant value 412 is too high, the processing system 102 may end up analyzing a reduced or ineffective number of possible sequences. For example, as the total number of base pairs in the TR indel variant approaches the phrase length 316, the number of available derived phrases and the likely occurrence of such mutations decrease. Accordingly, in some implementations, the indel variant value 412 in the range of three to five provides sufficient coverage for varying degrees of possible insertion and deletion mutations that are indicative of one or more types of cancer. This range of values may be sufficient to provide accurate results without requiring ineffective or inefficient amount of computing resources.

Additionally, the processing system 102 can further improve the efficiency and reduce the resource consumption using the segment length 320 (e.g., the length of the uniquely identifiable TR-based pattern). It has been found that the probability of mutation occurrences decreases as the tandem repeat segment length 320 is reduced. In particular, the mutation rate for genome TR sequences with segment length 320 of fewer than five base pairs is significantly less than genome TR sequences with the segment length 320 of five or more base pairs. Thus, the expected phrases 310 can be selected as the genome TR sequence with the segment length 320 of five or greater.

### Base Text Patterns - Storage/Tracking

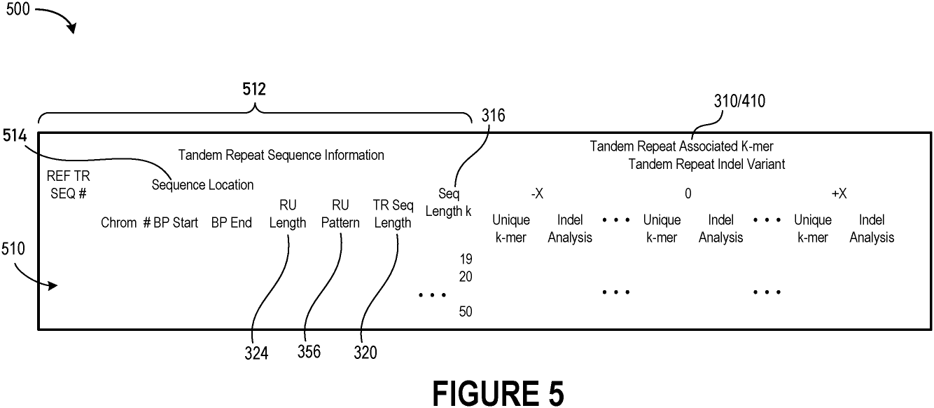

The processing system 102 can store the various phrases (e.g., the expected phrases 310 and/or the corresponding sets of the derived phrases 410) in the genome TR reference catalogue 230 (FIG. 2). FIG. 5 shows an example analysis template 500 in accordance with one or more implementations of the present technology. The processing system 102 can use the analysis template 500 to represent the various phrases and/or track the associated processing results. For example, the selected features, the PT input 166 (FIG. 1B), or a combination thereof can be provided according to the analysis template 500.

In some implementations, the analysis template 500 can correspond to a format for the genome TR reference catalogue 230. The genome TR reference catalogue 230 can include catalogue entries 510 for each instance of the unique segments 360 (e.g., uniquely identifiable or reference TR patterns). The entries 510 can include TR sequence information 512 that characterizes the unique segments 360and/or the derived segments 460. For example, the TR sequence information 512 can include a sequence location 514, the segment length 320, the base unit length 324, the repeated base unit 356, or a combination thereof.

The sequence location 514 can identify the location of the corresponding unique segment 360and/or expected phrase 310 within the reference genome. As an example, the sequence location 514 can be described based on the molecular location of the unique segment 360, such as (1) the chromosome on which the TR sequence is located and/or (2) the base pair numbers in the chromosome marking the beginning/end of the TR sequence. The sequence location 514 can act as a unique identifier that distinguishes one instance of the unique segment 360and/or the expected phrase 310 from another. For example, the expected phrases 310 that share the same repeated base unit 356and the base unit length 324 can be distinguished from one another based on the sequence location 514.

The entries 510 for each instance of the unique segment 360can include information for one or more instances of the corresponding phrases (e.g., expected and/or derived). For example, the entries 510 can include information for the expected phrases 310 and/or the derived phrases 410 with various values for the phrase length 316. For illustrative purposes, this instance of entries 510 is shown including information for the expected phrases 310 with phrase lengths corresponding from 19 base pairs to 50 base pairs. However, it is understood that the entries 510 can include information regarding fewer than 19 base pairs and/or more than 50 base pairs. As another example, the entries 510 can include information that distinguishes between the expected phrases 310 and the derived phrases 410. In some implementations, the entries 510 can identify the expected phrases 310 associated with a corresponding TR pattern. For instance, the TR pattern A8 beginning at position 10,513,372 can yield 16 sequences or expected phrases 310 having the phrase length 316 of 30 base pairs.

The entries 510 can further identify the derived phrases 410 that are absent from the reference genome. For illustrative purposes, Table 1 below summarizes the derived phrases 410 having the segment length 316 of 30 base pairs for the unique segment 360or TR pattern of “A8” beginning at position 10,513,372 (annotated as ‘372) on chromosome 22. In this example, each of the derived phrases 410 corresponding to indel variants with the indel variant value 412 ranging from “-5” to “+5” are not found in the reference genome.

TABLE 1

Chromosome 22, ‘372, “A8” Reference TR Associated Indel Phrase Summary

| | | |
|---|---|---|
| +5 | 16 | 16 |
| +4 | 17 | 17 |
| +3 | 18 | 18 |
| +2 | 19 | 19 |
| +1 | 20 | 20 |
| -1 | 22 | 22 |
| -2 | 23 | 23 |
| -3 | 24 | 24 |
| -4 | 25 | 25 |
| -5 | 26 | 26 |

The analysis template 500 can be used to track the statistical data generated during development/training of the ML model 104. For example, the processing system 102 can track the occurrences of certain mutations according to the sequence location 514 or the identifier for the corresponding entry 510 and the indel mutation offset/identifier. The processing system 102 can use the counted occurrences for each sample, each sample set, or a combination thereof to compute the correlation between the mutations and the onset of the corresponding type of cancer.

The analysis template 500 is shown for exemplary purposes as a template with a general layout for organizing information for each of the segments and/or phrases. It is understood that the analysis template 500 can include different categorizations and arrangements with additional or different pieces of information. Further, it is understood that an active or “in use” version of the genome TR reference catalogue 230 can be populated with values corresponding to the various categories of the entries 510.

### Control Flow

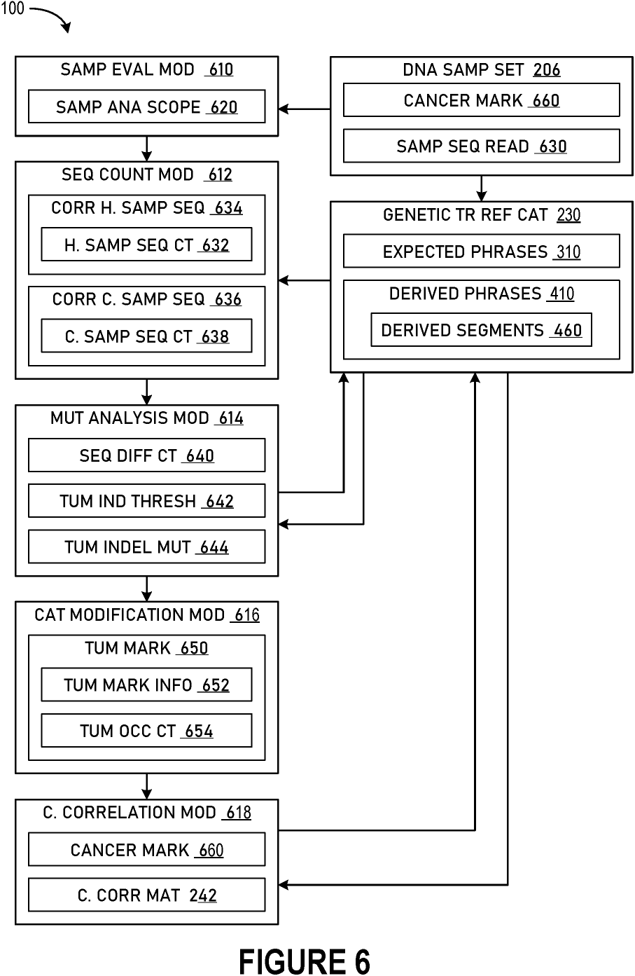

FIG. 6 shows a control flow diagram illustrating the functions of the computing system 100 in accordance with various implementations. The computing system 100 can be implemented to supplement and refine information in the genome TR reference catalogue 230 with information from the DNA sample sets 206 based on the unique segments 360 and the various phrases. In general, the computing system 100 can analyze one or more of the DNA sample sets 206 to process (1) mutations at specific locations of DNA sequences, (2) correlation of mutation patterns, (3) corresponding indications of one or more types of cancer, or a combination thereof. The functions of the computing system 100 can be implemented with a sample set evaluation module 610, a sequence count module 612, a mutation analysis module 614, a catalogue modification module 616, a cancer correlation module 618, or a combination thereof.

The evaluation module 610 can be configured evaluate the scope of the DNA sample set 206, including the cancer-free data 210, the non-regional data 211, and/or the cancer-specific data 212. For example, the evaluation module 610 can evaluate the DNA sample set 206 to identify factors, properties, or characteristics thereof to facilitate analysis of the different categories of data. In some implementations, the evaluation module 610 can be optional. The evaluation module 610 can generate a sample analysis scope 620 for the DNA sample set 206. The sample analysis scope 620 is a set of one or more factors that may govern/control the analysis of the DNA sample set 206. For example, the sample analysis scope 620 can be generated based on the supplemental information 220. The sample analysis scope 620 can be used to identify usable phrases (e.g., the expected phrases 310 and/or the derived phrases 410) based on the sequence location 514 and the phrase length k 316.

The computing system 100 can receive the derived phrases 410 and associated information from the genome TR reference catalogue 230 and/or the DNA sample set 206. The mutation analysis mechanism can be implemented with the count module 612 and the analysis module 614. The count module 612 may be responsible for calculating a number of occurrences (e.g., a sequence count) for specific DNA sequences/phrase in a sample set. The count module 612 can calculate the sequence count based on a number of sample sequence reads 630, such as the sequence reads for the DNA fragments in one or more categories of data in the DNA sample set 206.

For the cancer-free data 210, the count module 612 can calculate a healthy sample sequence count 632 for each instance of a corresponding healthy sample sequence 634 identified in the cancer-free data 210. The corresponding healthy sample sequence 634 is a DNA sequence in the healthy sample DNA information 634 that corresponds to one of the derived segments 460 and/or the derived phrases 410. The heathy sample sequence count 632 is the number of times that the corresponding healthy sample sequence 634 is identified in the cancer-free data 210. Similarly, for the cancer-specific data 212 and/or the non-regional data 211, the count module 612 can calculate count values for each instance of a targeted sequence identified in the data group. In other words, the count module 612 can calculate the number of times the various phrases are found within the samples according to the corresponding groups/categories.

The count module 612 can identify the corresponding healthy sample sequence 634 and the corresponding cancerous sample sequence 638 for a given expected phrase, and more specifically the derived phrase. For example, the sequence count module 612 can search through the different categories of data for matches to one or more of the derived segments within the corresponding phrases. As one specific example, the count module 612 can search for a string of consecutive base pairs that matches one of the derived segments 460 of the derived phrases 410.

The count module 612 can calculate the healthy sample sequence count 632 as the total number of each of the corresponding healthy sample sequence 634 identified in each of the sample sequence reads 630 in the cancer-free data 210. In many cases, the corresponding healthy sample sequence 634 will correspond with a single instance of the tandem repeat indel variants 310. In these cases, the total value of the healthy sample sequence count 632 will be equal to the total number of the sample sequence reads 630 in the cancer-free data 210. For example, where the cancer-free data 210 includes 40 instances of the sample sequence reads 630 per DNA segment, the healthy sample sequence count 632 for a given instance of the corresponding healthy sample sequence 634 should also be 40. The case of non-unity between the number of sequencing reads and the healthy sample sequence count 632 can generally be attributed to sequencing errors.

In many cases, the corresponding healthy sample sequence 634 will match with the phrase with the indel variant value 312 of zero (e.g., the expected phrase with no insertions or deletions of the unique segment 360). However, in some cases, the corresponding healthy sample sequence 634 can differ. The differences between the corresponding healthy sample sequence 634 and the phrase with the indel variant value 312 of zero can account for wild type variants (e.g., naturally occurring variations) in the cancer-free data 210.

Similarly, the count module 612 can calculate the cancerous sample sequence count 636 for each of the corresponding cancerous sample sequence 638 that appear in the sample sequence reads 630 in the cancer-specific data 212. Due to possible mutations, the cancer-specific data 212 can include multiple different instances of the corresponding cancerous sample sequence 638 matching different instances of the derived segments 460, with each corresponding cancerous sample sequence 638 having varying values of the cancerous sample sequence count 636. As an example, in some cases, the corresponding cancerous sample sequence 638 and cancerous sample sequence count 636 will match with the corresponding heathy sample sequence count 634 and healthy sample sequence count 632, indicating no mutations. As another example, for a given instance of the derived phrase 410, the cancer-specific data 212 may have a split in the cancerous sample sequence count 636 between the cancerous sample sequence 638 that is the same as the corresponding healthy sample sequence 634 and one or more other instances of the tandem repeat indel variants 310. For a given instance of the derived phrase 410, the count module 612 can track the cancerous sample sequence count 636 for each different instance of the corresponding cancerous sample sequence 638 in the cancer-specific data 212.

The flow can continue to the analysis module 614. The analysis module 614 may be responsible for determining whether a mutation exists in the corresponding cancerous sample sequence 638 of the cancer-specific data 212. In general, the existence of a mutation in the cancer-specific data 212 can be determined based on differences in the repeated TR patterns between the corresponding heathy sample sequence 634 and the corresponding cancerous sample sequence 638. More specifically, a difference in the number of the repeated base unit 356 can represent the existence of an indel mutation (e.g., a mutation corresponding to an insertion or a deletion of the repeated TR unit), such as for cancer-specific data 212 in comparison to the cancer-free data 210. For example, the analysis module 614 can determine that a mutation exists when the corresponding cancerous sample sequence 638 matches one of the derived segments 460 and/or the derived phrases different from that of the corresponding healthy sample sequence 634. In another example, the analysis module 614 can determine the difference between the corresponding healthy sample sequence 634 and the corresponding cancerous sample sequence 638 based on a sequence different count 640 (e.g., the total number of corresponding cancerous sample sequences 638 differing from the corresponding healthy sample sequences 634). In the case where the sequence difference count 640 indicates no differences, such as when the sequence difference count 640 is zero, the analysis module 614 can determine that no mutation exists in the corresponding cancerous sample sequence 638.

In general, the analysis module 614 can determine that an indel mutation has occurred when the sequence difference count 640 is a non-zero value. In some implementations, the analysis module 614 determines whether the indel mutation is a tumorous indel mutation based on whether the sequence difference count 640 is greater than the error percentage of the approach or apparatus used to sequence the cancer-free data 210, cancer-specific data 212, or a combination thereof.

In another implementation, the analysis module 614 can determine whether the indel mutation is a tumorous indel mutation 644 based on a tumor indication threshold 642. The tumor indication threshold 642 is an indicator of whether the number of mutations for a particular sequence in the cancer-specific data 212 indicates the existence of a tumorous indel mutation 644. The tumorous indel mutation 644 may occur when the sequence difference count 640 exceeds a tumor indication threshold 642. As an example, the tumor indication threshold 642 can be based on a percentage between the total number of sample sequence reads 630 and the sequence difference count 640. As a specific example, the tumor indication threshold 642 can require a sequence different count 640 to be greater than 60 percent of the sample sequence reads 630 for the cancer-specific data 212. In another specific example, the tumor indication threshold 642 can require the sequence difference count 640 to be greater than 80 percent of the sample sequence reads 630 for the cancer-specific data 212. In another specific example, the tumor indication threshold 642 require the sequence difference count 640 to be greater than 90 percent of the sample sequence reads 630 for the cancer-specific data 212.

When the corresponding cancerous sample sequence 638 includes the tumorous indel mutation 644, the computing system 100 can implement the modification module 616 to update or modify the genome TR reference catalogue 230. Said another way, the computing system 100 can implement the modification module 616 responsive to determining that the corresponding cancerous sample sequence 638 includes the tumorous indel mutation 644. For example, the modification module 616 can modify the genome TR reference catalogue 230 by identifying the instance of the catalogue entries 510 as a tumor marker 650 when the tumorous indel mutation 644 exists in the corresponding cancerous sample sequence 638.

The catalogue entries 510 that are identified as a tumor marker 650 can be modified by the modification module 616 to include tumor marker information 652. Some examples of the tumor marker information 652 can include a tumor occurrence count 654, such as the number of times that the tumorous indel mutation 644 was identified in a particular instance of the segment/phrase (e.g., TR pattern) for a given form of cancer. As a specific example, the tumor occurrence count 654 can be compiled from analysis for the DNA sample sets 206 for numerous cancer patients.

In another example, the tumor marker identification 652 can include information about the different instances of the corresponding cancerous sample sequence 638 matching to different instances of the derived segments/phrases along with the cancerous sample sequence count 636, the total number of sample sequence reads 630 of the DNA sample set 206, all or portions of the supplemental information 220, or a combination thereof. In a further example, the tumor marker information 652 can include the number of repeated base units 356in the corresponding cancerous sample sequence 638 that were different from the corresponding healthy sample sequence 634.

The tumor marker information 652 can include information based on the supplemental information 220. For example, the tumor marker information 652 can include the supplemental information 220 (e.g., source information), such as the cancer type, the stage of cancer development, organ or tissue from which the sample was extracted, or a combination thereof. In another example, the tumor marker information 652 can include the supplemental information 220 of the patient demographic information, such as the age, the gender, the ethnicity, the geographic location of where the patient resides or has been, the duration of time that the patient stayed or resided at the geographic location, predispositions for genetic disorders or cancer development, or a combination thereof.

The computing system 100 can use one or more instances of the segments/phrases identified as the tumor marker 650 to generate the cancer correlation matrix 242 with the correlation module 618. For example, the correlation module 618 can identify cancer markers 660 based on the tumor occurrence count 654 for each of the tumor markers 650 in the genome TR reference catalogue 230. The cancer markers 660 can correspond to mutation hotspots that are specific to indel mutations in instances of the TR patterns. In one implementation, the correlation module 618 can identify the cancer markers 660 based on regression analysis. For example, the regression analysis can be performed with a receiver operating characteristic curve to the optimum sensitivity and specificity from the tumor markers 650, tumor occurrence count 654, or a combination thereof to determine the cancer markers 660.

In another implementation, the correlation module 618 can identify the cancer markers 660 based on a ratio between, or percentage of, the tumor occurrence count 654 for the tumor marker 650 and the total number of the DNA sample sets 206 of a particular form of cancer that have been analyzed for the tumor marker 650. As a specific example, the correlation module 618 can identify the cancer markers 660 as the tumor markers 650 when the ratio between the tumor occurrence count 654 and the total number of DNA sample sets 206 that are analyzed is 90 percent or more of the DNA sample sets 206 for a particular form of cancer. In this case, the cancer correlation matrix 242 can include the cancer markers 660 that were identified in this manner.

In a further implementation, the correlation module 618 generates the cancer correlation matrix 242 as the tumor markers 650 that are common among a percentage of the DNA sample sets 206 for a particular form of cancer are found. For example, the correlation module 618 can generate the cancer correlation matrix 242 as the tumor markers 650 appear in 90 percent or more of the total number of DNA sample sets 206. In other implementations, the correlation module 618 can generate the cancer correlation matrix 242 through other methods, such as regression analysis or clustering.

The correlation module 618 can generate the cancer correlation matrix 242 taking into account the supplemental information 220, such as the patient demographic information, to generate the cancer correlation matrix 242 for subpopulations. For example, the correlation module 618 can generate the cancer correlation matrix 242 based on the patient demographic information specific to gender, nationality, geographic location, occupation, age, another characteristic, or a combination of characteristics.

The computing system 100 has been described in the context of modules that perform, serve, or support certain functions as an example. The computing system 100 can partition or order the modules differently. For example, the evaluation module 610 could be implemented on the processing system 102, while the count module 612, analysis module 614, and correlation module 618 could be implemented on an external device. Alternatively, the processing system 102 can include the various modules described above.

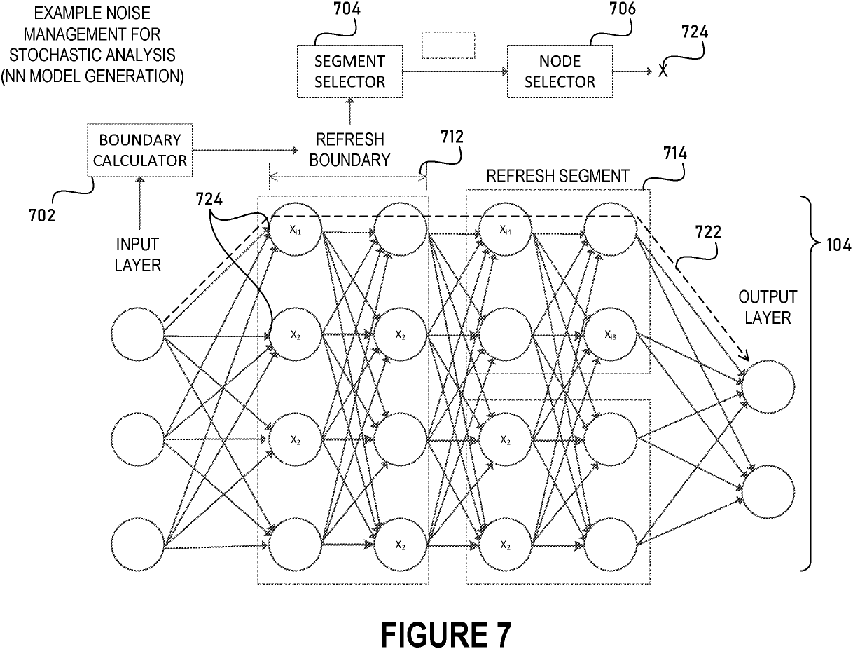

FIG. 7 shows an example implementation of the noise mitigation mechanism 105 of FIG. 1 in accordance with one or more implementations of the present technology. For the purpose of illustration, the example implementation is described in the context of a stochastic ML model. As mentioned above, stochastic ML models can exhibit different behaviors on different runs through the underlying computational architecture during training. As such, it may be impractical to implement a simple heuristic for determining the computational locations/timings at which to implement the noise mitigation mechanism 105. Simply put, because the computational architecture is malleable during training, a simple heuristic that specifies where to implement the noise mitigation mechanism 105 may have limited value.

For that reason, the processing system 102 (FIGS. 1A/1B) may implement a dynamic approach for identifying the computational locations/timings at which to implement the noise mitigation mechanism. Thus, the processing system 102 can select different points within the overall analysis and coordinate implementation of the noise mitigation mechanism 105 at the selected points. The processing system 102 can randomly or pseudo-randomly select computation locations/timings at which to deploy the noise mitigation mechanism 105 within the ML model 104. Consider, for example, FIG. 7 in which the ML model 104 is a neural network comprised of nodes at which functions are executed. At selected nodes, the processing system 102 can implement the noise mitigation mechanism 105, such as the bootstrapping process that corresponds the HE scheme, so as to limit the noise that percolates or permeates through the neural network.

In some implementations, the processing system 102 can include a boundary calculator 702, a segment selector 704, and/or a node selector 706. The boundary calculator 702, the segment selector 704, and the node selector 706 may be implemented in software, hardware, firmware or a combination thereof. As further discussed below, the processing system 102 can use the boundary calculator 702, the segment selector 704, and/or the node selector 706 to selectively implement the noise mitigation mechanism 105.

The boundary calculator 702 can be configured to calculate a refresh boundary 712 based on one or more aspects of the input CT data (e.g., data size, encoding detail/complexity, and the like) and/or the analysis tool (e.g., a number of inputs/outputs, a number of nodes/tiers, an analysis type, and the like). The refresh boundary 712 can represent the threshold number of computations that are allowable for maintaining reversible levels of noise. In some implementations, the boundary calculator 702 may calculate the refresh boundary 712 offline or before generating, training, and/or implementing the ML model 104. As an example, for a given ML model, the boundary calculator 702 could calculate the refresh boundary 712 prior to implementation using a testing dataset (also referred to as a “validation dataset”). The validation dataset may be constructed, structured, or fed into the ML model 104 in such a manner that the outputs of certain computation locations (e.g., nodes) are known, and therefore error throughout the ML model 104 can be monitored.

The segment selector 704 can be configured to determine groupings for expected calculations based on the refresh boundary 712. For example, the segment selector 704 can map the refresh boundary 712 to computational location (e.g., a layer, a step, a single node, a grouping of multiple nodes, etc.) of the ML structure. By mapping the refresh boundary 712 to a computational location of the ML model 104, the segment selector 704 may define a refresh segment 714 as shown in FIG. 7. The refresh segment 714 may be representative of a grouping of layers, steps, nodes, or the like within which the noise mitigation mechanism 105 is to be employed. In other words, the processing system 102 can select one or more computational points within the refresh segment 714 to implement the noise refresh segment 105 such that the overall data or a targeted portion thereof is guaranteed to be refreshed by the end of the refresh segment 714. Also, the segment selector 704 can determine the groupings to have sizes less than the refresh boundary 712 and/or have randomly selected sizes. In some implementations, the segment selector 704 can further divide the data/computations into subgroupings within the refresh boundary 712.

The node selector 706 can be configured to assign/schedule implementation or triggering of the noise mitigation mechanism 105 within the refresh boundary 712 and/or within the refresh segment 714. The node selector 706 can select the computational locations at least pseudo-randomly. In some implementations, the node selector 706 can use LFSR with prime polynomials for the random selection. Additionally or alternatively, the node selector 706 can follow one or more predetermined rules to select the refresh locations after an initial set of random selections. These predetermined rules may be representative of heuristics that, when programmed in the processing system 102, can ensure that at least one data refresh occurs along a computation path. In other words, the node selector 706 can iteratively select the nodes based on considering the previous selections, thus successively reducing the randomness across the iterations.

In some implementations, the processing system 102 can operate the segment selector 704 and the node selector 706 iteratively. For example, the segment selector 704 can begin from one end of a computational chain 722 and generate a first set of one or more groupings within the refresh boundary 712. The node selector 706 can select the nodes that will implement the noise mitigation mechanism 105. In the next iteration, the segment selector 704 can apply the refresh boundary 712 to one or more of the nodes/refresh locations selected in the previous iteration to identify the new segments. Accordingly, the processing system 102 can ensure that noise mitigation occurs before the noise reaches unmanageable levels.

The processing system 102 can adjust or add to the refresh locations after determining all of the segments. The processing system 102 can analyze data flows or computation paths to determine whether refreshes occur within the threshold number of computations. When the path segments exceed the threshold number, the processing system 102 can designate/schedule additional refreshes therein.

The processing system 102 can implement the boundary calculator 702, segment selector 704, and/or the node selector 706 during the generation/training of the ML model and/or during implementation of the ML model. For example, the processing system 102 can use HE cipher training data to generate or train the ML model. During such modeling processes, the processing system 102 can implement the boundary calculator 702, the segment selector 704, and/or the node selector 706 as described above. The processing system 102 can implement the boundary calculator 702, the segment selector 704, and/or the node selector 706 in conjunction with trained models. In other words, the processing system 102 can generate the ML models to include the boundary calculator 702, the segment selector 704, and/or the node selector 706 or integrate them into the ML models. Accordingly, the processing system 102 can selectively and randomly implement the noise mitigation mechanism 105 while using the trained ML model to generate/derive the result (ciphertext) data.

As an illustrative example, the source device 102 of FIG. 1 can be configured to encode the data using a matrix-oriented HE scheme (e.g., MORE) and a predetermined data size/format. The processing system 102 can be configured to generate and/or implement an ML model that utilize matrix operations (e.g., a neural network model) to generate the output result (e.g., the encoded result 134 (FIG. 1B)). The processing system 102 can derive the number of matrix operations across the computation paths 722 within the ML model 104 based on the predetermined data size/format of the input CT data. The processing system 102 can use the derived or tracked number of operations to segment the path and implement the refresh functions within the segments as described above. Thus, the processing system 102 can ensure that the resulting segments remain bootstrappable and that the final output result remains decipherable.

While the implementations shown in FIG. 7 is described in the context of a stochastic ML model, the approach may be similarly applicable to deterministic ML models. As mentioned above, deterministic ML models are models that, when given a certain input, will not only always produce the same output, but also will pass through the underlying computational architecture in the same way. This makes training of deterministic ML models much more predictable than training of stochastic ML models for this reason.

One benefit of deterministic ML models is that the underlying computational architecture generally does not change during training. While aspects (e.g., weights) may change as part of training, the computational architecture itself will remain largely, if not entirely, the same. Because the computational architecture remains the same, the processing system 102 may implement a more straightforward procedure for identifying computational locations at which to implement the noise mitigation mechanism.

As an example, the node selector 706 may assign/schedule implementation of the noise mitigation mechanism 105 in accordance with one or more predetermined rules. These predetermined rules may specify that the noise mitigation mechanism 105 should be implemented at a certain frequency (e.g., every n operations, where n is an integer larger than one).

As another example, the node selector 706 may select an arbitrary subset of computational locations at which the noise mitigation mechanism 105 could be implemented. Then, the processing system 102 could provide a validation dataset to the deterministic ML model as input - with the noise mitigation mechanism 105 implemented at the (arbitrarily) selected computational locations - and then examine the outputs produced at various computational locations. By examining the error at these various computational locations, the processing system 102 may be able to “tune” the deterministic ML model by iteratively selecting different computational locations at which to implement the noise mitigation mechanism 105.

Note that more complicated procedures for identifying computational locations 724 at which to implement the noise mitigation mechanism could still be employed by the node selector 706. For example, the node selector 706 could randomly or pseudo-randomly select the computational locations (e.g., using LFSR with prime polynomials) as discussed above. However, because a deterministic ML model will offer greater insight into its inner workings, these more complicated procedures may not be necessary to ensure that noise is sufficiently mitigated or addressed.

### Exemplary Methodology For Inhibiting Error Propagation During Training

Significant development has occurred in the realm of computer-implemented tasks that are facilitated through ML. As an example, healthcare systems have begun routinely using ML models to examine patient health data to derive insights that useful for diagnostic purposes, treatment purposes, etc. However, a single entity is rarely responsible for managing the patient health data throughout this entire process. Imagine, for example, that a healthcare system is interested in having patient health data examined by an analysis service for the purpose of rendering diagnoses. In such a scenario, the healthcare system may provide the patient health data to the analysis service that manages the ML models to be applied thereto. After the ML models have been applied to the patient health data, the analysis service may provide the outputs produced by the ML models — or analyses of the outputs — to the healthcare system.

There are several downsides to this approach. First, sensitive information — like the patient health data and outputs produced by the ML models — could be inadvertently provided to an unauthorized person or entity as part of the transfer process. Second, because the healthcare system and analysis service have handled the sensitive information, the likelihood of unauthorized access (e.g., a data breach) is higher than it would be if the sensitive information were handled by a single entity.

To mitigate these downsides, the analysis service could employ a computing system that utilizes HE to obfuscate the patient health data. In fact, if the ML models have been trained to perform computations on CT, then the patient health data obtained from the healthcare system could be in CT and the outputs produced by the ML models could be in CT. Accordingly, the analysis service may only handle sensitive information in its encrypted form, thereby lessening the risks of the aforementioned downsides.

There is an issue with utilizing ML models that have been homomorphically encrypted to analyze patient health data that has been homomorphically encrypted, however. Assume, for example, that the ML model to be applied to the patient health data - in its encrypted form — is a neural network. For neural networks, there is a matrix multiple and add whenever a node performs an operation. Some noise will be generated whenever this occurs, and at some level of the neural network, noise may become enough of an issue that the prediction is affected. For this reason, noise should be managed while the neural network is being trained.

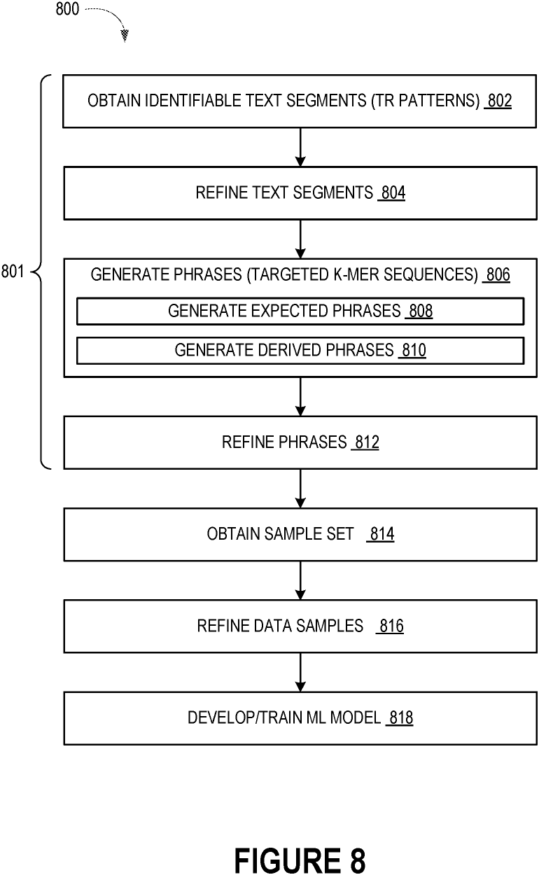

FIG. 8 shows a flow chart of a method 800 for processing and refining DNA-based text data for cancer analysis in accordance with one or more implementations of the present technology. The method 800 can be implemented using the computing system 100 (FIG. 1A) including the processing system 102 (FIG. 1A). The method 800 can be for developing the ML model 104 (FIG. 1) including generating the various phrases and refining the processing results (via, e.g., the refinement mechanism 115 (FIG. 1)) as described above.

The method 800 includes the computing system 100 obtaining identifiable text sequences (e.g., TR-based patterns) at block 802. In some implementations, the processing system 102 can obtain the identifiable text sequences based on generating the unique segments 360 (FIG. 3) from the reference data 112 (FIG. 1A), such as by generating the character patterns representative of the identifiable TR patterns the human genome. In other implementations, the processing system 102 can access/receive the unique segments 360 generated by an external system/device.

The obtained unique segments 360 can serve as an initial set of segments representative of TR sequences. Each segment in the initial set can include N number of adjacently repeated base units 356. The repeated base units 356 for the initial set can have the base unit length 324 that is uniform across the segments.

At block 804, the computing system 100 can refine the identifiable text segments, such as by using/implementing the consecutive overlap filter 252 (FIG. 2). In some implementations, the processing system 102 can refine the identifiable text segments by removing the overlaps 352 (FIG. 3A), such as the TR patterns that are consecutive of and/or overlap each other, from the initial set of the unique segments 360 as described above. The processing system 102 can generate the segments based on removing the overlaps 352 from the initial set.

At block 806, the computing system 100 can generate the phrases, such as the k-mer sequences targeted for use in subsequent data processing. For example, at block 808, the processing system 102 can generate the expected phrases 310 (FIG. 3). The processing system 102 can use the unique segments 360 (e.g., uniquely identifiable TR patterns) to generate the expected phrases 310, such as by adding different combinations of the flanking text 314 (FIG. 3) as described above. Also, at block 810, the processing system 102 can generate the derived phrases 410 (FIG. 4). The processing system 102 can use the expected phrases 310 to generate the derived phrases 410, such as by adjusting the unique segments 360 within the expected phrases to the derived segments 460 representative of indel mutations as described above.

In some implementations, the generated phrases can serve as an initial set. The generated phrases can correspond to different locations within the human genome. For example, the phrases can have the phrase length k 316 and include (1) location-specific TR-based segments (e.g., expected phrases 310) and/or (2) indel derivations of the TR-based segments adjacent to corresponding sets of flanking texts (e.g., derived phrases 410).

At block 812, the computing system 100 can refine the set of phrases, such as by using/implementing the duplicate filter 254 (FIG. 2). For example, the processing system 102 can refine the expected phrases 310 and/or derived phrases 410 by removing the duplicates or representations of DNA sequences or mutations that may correspond to more than one location. In other words, the processing system 102 can search for inadvertently generated representations of mutations that match mutations or expected/healthy sequences corresponding to a different location in the human genome as described above.

The operations described above for one or more of the blocks 802-812 can correspond to a block 801 for generating text phrases that represent different DNA sequences. The generated text phrases can represent various uniquely identifiable DNA sequences and mutations sequences for TR indel variants. The generated/refined text phrases can be used to determine correlations between the various mutations and onset cancer in the DNA sample set 206.

At block 814, the computing system 100 can obtain one or more sample sets (e.g., the DNA sample set 206 (FIG. 2)). In some implementations, the processing system 102 can receive sequenced DNA data from publicly available databases, healthcare providers, and/or submitting patients. The obtained data sample sets can include corresponding or known diagnoses, such as categorizations or tags identifying that the DNA data is from patients confirmed to be without cancer or confirmed to have specific cancers. Additionally, the obtained data can include physiological source locations of the DNA data. For samples sourced from the patients having cancer, the source locations can be the cancerous tumor or a location different from or unrelated to the malignant tumors. Accordingly, the processing system 102 can include a combination of the cancer-free data 210, the non-regional data 211, and the cancer-specific data 212, illustrated in FIG. 2. The obtained DNA sample set 112 can further include other details, such as the supplemental information 220 (FIG. 2), the sample read depth 214 (FIG. 2), the sample quality score 216 (FIG. 2), or the like.

At block 816, the computing system 100 can refine the data samples 816, such as by using/implementing the quality filter 256 (FIG. 2). For example, the processing system 102 can identify the characters corresponding to nucleotides having phred scores less than the quality threshold. The processing system 102 can replace the identified characters with a predetermined dummy letter as described above. Additionally or alternatively, the processing system 102 can filter and/or adjust for nonuniform read counts or read depths across the DNA sample set 206. The processing system 102 can remove sample data having the sample read depth 214 below a depth requirement/threshold as described above. The processing system 102 can also adjust for the nonuniformity by calculating and applying the scale factor to the read counts as described above.

At block 818, the computing system 100 can develop and train the ML model 104 using the refined phrases and the refined data samples. For example, the processing system 102 can count and analyze the various somatic mutations, compute correlations between the mutations and cancers, and the like as described above. Using the results, the processing system 102 can select a set of features that include phrases having sufficient correlations to one or more types of cancers. The processing system 102 can design and train the ML model 104 using the selected features (e.g., correlative phrases representative of cancer-causing somatic mutations).

In developing and training the ML model 104, the processing system 102 can further refine the intermediate processing results. For example, the processing system 102 can correct for comparison noises using the p-value criteria. Also, the processing system 102 can refine the intermediate results per the fractional features. The processing system 102 can use a fraction filter in classifying or distinguishing between somatic and non-somatic mutations.

The processing system 102 can develop/train the ML model 104 such that the model is configured to compute a cancer signal based on analyzing text-based patient DNA data according to represented somatic indel mutations in patient DNA. The processing system 102 can develop/train the ML model 104 based on computing correlations between mutations (as represented by the derived phrases) and onset/existence of one or more types of cancers as represented by the DNA sample set 206. Using the correlations, the ML model 104 can be configured to compute the cancer signal that represents (1) a likelihood that a corresponding patient has developed the one or more types of cancer, (2) a likelihood that the patient will develop the one or more types of cancer within a given duration, (3) a development status at least leading up to onset of one or more types of cancer, or a combination thereof.

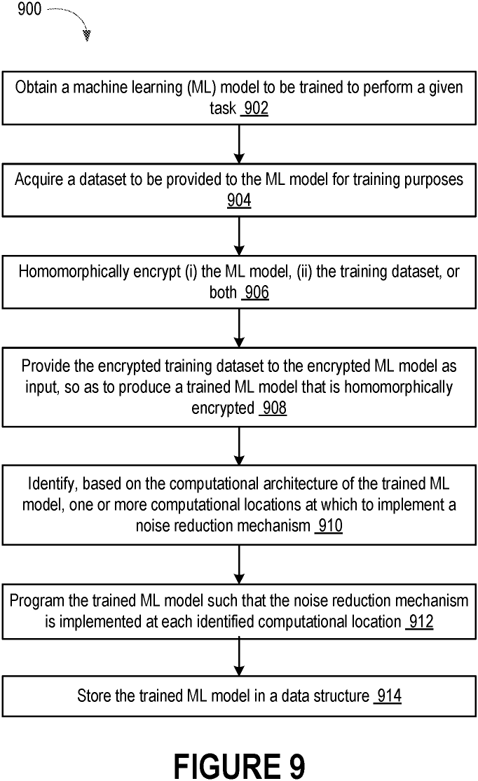

FIG. 9 shows a flow chart of an example method 900 for configuring the cancer analysis (e.g., the method 800 (FIG. 8)) to process encoded data with noise management in accordance with one or more implementations of the present technology. For the purpose of illustration, the method 900 is described as being performed by the processing system 102 (FIG. 1A). Initially, the processing system 102 can obtain an ML model (e.g., the model 104 (FIG. 1A) or an initial version thereof) to be trained to perform a given task as illustrated at block 902. In some embodiments the ML model is stochastic, while in other embodiments the ML model is deterministic. While the nature of the ML model may influence aspects of the method 900, the general approach may be largely the same.

Moreover, the nature of the ML model is not particularly limited. For example, the ML model could be a multiclass classification model (also referred to as a “multiclass classifier” or “classifier”) that is able to classify a patient among multiple diseases based on analysis of corresponding health data. The corresponding health data could include genetic information (e.g., a sequence listing corresponding to a sample), symptom information, and other information obtained from the electronic health record (also referred to as the “electronic medical record”) of the patient.

The processing system 102 can then acquire a dataset to be provided to the ML model for training purposes as illustrated at block 904. The content of the dataset will depend on the intended application of the ML model. If, for example, the ML model is to be trained to classify patients among multiple diseases based on an analysis of genetic information, then the training dataset can include genetic information for various individuals who are known have the multiple diseases. Accordingly, the training dataset may include genetic information for one or more individuals who are known to have a first disease of the multiple diseases, genetic information for one or more individuals who are known to have a second disease of the multiple diseases, etc.

Then, the processing system 102 can homomorphically encrypt (i) the ML model, (ii) the training dataset, or both as illustrated at block 906. At a high level, this can be done to allow the ML model to learn how to perform computations on an input in encrypted form, such as the CT evaluation target 132 (FIG. 1B). Said another way, the processing system 102 may perform HE to ensure the ML model learns how to perform computations on CT without requiring the corresponding PT. HE can provide operability by representing the computations as Boolean or arithmetic circuits, such as by using different amounts, types, and/or layers of logical gates (e.g., AND, OR, NOT, etc.). Examples of HE include lattice-based cryptography, Benaloh cryptosystem, multivariate quadratic FHE, Paillier encryption scheme, ElGamal encryption scheme, MORE scheme, and the like. Thereafter, the processing system 102 can provide the encrypted training dataset to the encrypted ML model as input, so as to produce a trained ML model that is still homomorphically encrypted as illustrated at block 908.

ML models that have been homomorphically encrypted can struggle with errors that propagate through the underlying computational architecture. Accordingly, the processing system 102 may identify, based on the computational architecture of the trained Ml model, one or more computational locations at which to implement a noise reduction mechanism (e.g., the noise management mechanism 105 (FIG. 1B)) as illustrated at block 910. This can be accomplished in various ways as discussed above. For example, the processing system 102 can identify the boundaries and/or the segments, and then randomly or pseudo-randomly select computational locations within the computational architecture using a selection mechanism (e.g., LSFR based on prime polynomials) as described above. At a high level, the selection mechanism may function as a random or pseudo-random number generator. In some implementations, outputs produced by the selection mechanism are homomorphically encrypted, while in other embodiments the selection mechanism is homomorphically encrypted. As another example, the processing system 102 could select computational locations within the computational architecture in accordance with one or more predetermined rules.

The processing system 102 can then program the trained model such that the noise reduction mechanism 105 is implemented at each identified computational location as illustrated at 912. This approach allows for random (or pseudo-random) yet consistent management of noise, which ensures that noise does not propagate through the computational architecture during runtime. The number of computational locations at which the noise reduction mechanism is implemented may depend on the approach used to identify those computational locations. For example, if the processing system 102 employs a selection mechanism that is implemented as an LFSR that acts as a primitive polynomial, then the number of computational locations may be based on the number of the primitive polynomial. Regardless of how the computational locations are identified, the goal may be to guarantee that different hierarchies (e.g., levels of nodes in the case of a neural network) are sufficiently selected to ensure that the output of each hierarchy comes out “clean” without significant noise.

Note that selection of the computational locations may be constrained in some embodiments. For example, the processing system 102 may be programmed to ensure that less than half of the computational locations associated with each hierarchy are selected for implementation of the noise reduction mechanism. Thus, if the trained ML model is a neural network, then the processing system 102 may ensure that less than half of the nodes on each layer are selected. This may be done to ensure that, in the event the noise reduction mechanism decrypts the encrypted data, less than half of the nodes are decrypting in any given layer of the neural network.

At block 914, the processing system 102 can then store the trained ML model in a data structure in preparation for future deployment. For example, the processing system 102 may store the trained ML model 104 in a data structure and then encode information (e.g., in the form of metadata) regarding the multiple diseases, training dataset, or the like in the data structure.

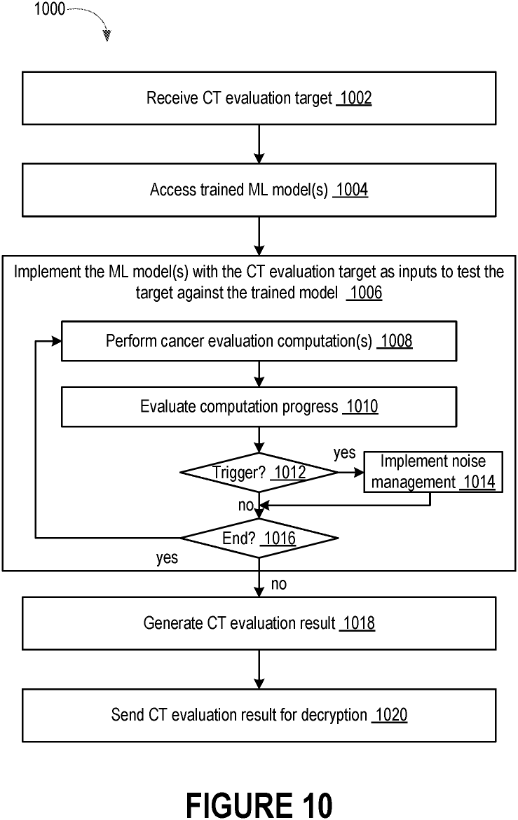

FIG. 10 shows a flow chart of an example method 1000 for implementing the trained model (e.g., the ML model 104 (FIG. 1B)) with noise management in accordance with one or more implementations of the present technology. For the purpose of illustration, the method 900 is described as being performed by the processing system 102 (FIG. 1B).

At block 1002, the processing system 102 can receive a CT evaluation target (e.g., the CT evaluation target 132 (FIG. 1B)) from the sourcing device (FIG. 1B). The received target can be in CT as a homomorphically encoded representation of the PT input 166 (FIG. 1B) that includes text-based representation of a patient’s DNA information. The PT input 166 can correspond to a DNA sequencing result of a biological sample (e.g., tissue, cheek swab, blood, saliva, or the like), and the CT evaluation target can include that corresponding encoded representation that is submitted for evaluation.

At block 1004, the processing system 102 can access one or more trained ML models appropriate for the CT evaluation target. The processing system 102 can access the ML models that have been trained according to the targeted phrases and using the noise management mechanism 105 (FIG. 1B) as described above.

The processing system 102 can select and access one or more of the trained ML models 104 (FIG. 1B) that is configured to evaluate the received sample, such as according to a description or a request accompanying the data. For example, the processing system 102 can access a multi-class classifier model or a set of models that simultaneously tests for multiple types of cancers, such as for cancer screening requests. Also, a healthcare provider can specify one or more specific types of cancer for the submitted data, and the processing system 102 can select one or more models that are configured to test for the requested types of data.

At block 1006, the processing system 102 can implement the accessed ML models with the CT evaluation target as inputs. Accordingly the processing system 102 can test the evaluation target against the trained ML model configured to predict existence of certain types of cancers, likely future onset thereof, recurrence thereof, or other related statuses. The processing system 102 can implement the model by iteratively performing the cancer-based computations while managing the internally-generated noise.

At block 1008, the processing system 102 can perform the cancer evaluation computation(s). Using the neural network example of FIG. 7, the processing system 102 can provide the CT evaluation target 132 into the input nodes of the ML model 104. The processing system 102 can perform the corresponding computations.

At block 1010, the processing system 102 can evaluate the computation progress. For example, the processing system 102 can identify the location of the next computational node, track and evaluate the computation count 126 (FIG. 1A), or the like.

At decision block 1012, the processing system 102 can determine whether to trigger the noise management mechanism 105 (e.g., a trigger timing) based on the computation progress. In some implementations, the processing system 102 can perform the trigger determination based on following fixed computational paths that have predetermined trigger locations/timings. In other implementations, the processing system 102 can implement a pseudo-random selector within corresponding boundaries and segments to select the timing/location as described above.

When triggered, the processing system 102 can implement the noise management mechanism 105. For example, the processing system 102 can implement the bootstrapping algorithm or other similar noise reduction scheme on all intermediate data at the time of trigger. Also, the processing system 102 can implement the noise management mechanism 105 at the selected node or timing for a corresponding subset of intermediate results, thereby iteratively refreshing the data in portions. When the trigger is not selected, the noise management mechanism 105 can be bypassed.

Subsequently, the processing system 102 can determine whether the computations for the ML model 104 is at an end as illustrated at decision block 1016. The processing system 102 can iteratively perform the computations and the noise management as illustrated in blocks 1008-1016 until the end of the model computations. When the computations end, the processing system 102 can generate cancer signature (e.g., the evaluation results 134 (FIG. 1B)) according to the computations as illustrated at block 1018. Given the CT input and CT-preserving operations (e.g., HE operations), the evaluation results 134 can be in encoded or CT format. As described above, the generated evaluation results 134 can include CT data representative of (1) a likelihood that a corresponding patient has developed one or more types of cancer, (2) a likelihood that the patient will develop the one or more types of cancer within a given duration, (3) a development status at least leading up to onset or recurrence of the one or more types of cancer, or a combination thereof.

At block 1020, the processing system 102 can send the CT evaluation results 134 to the sourcing device 152. The CT evaluation results 134 can be decrypted at the sourcing device 152 to provide PT results indicating a likely existence or absence of one or more types of cancer, a likely onset thereof in the future, a progress of developing/treating the one or more types of cancer, or other similar statuses regarding the one or more types of cancer. The CT evaluation results 134 can be communicated to and decrypted at the sourcing device 152 to preserve the patient privacy regarding the analysis and the corresponding results.

### Computing System

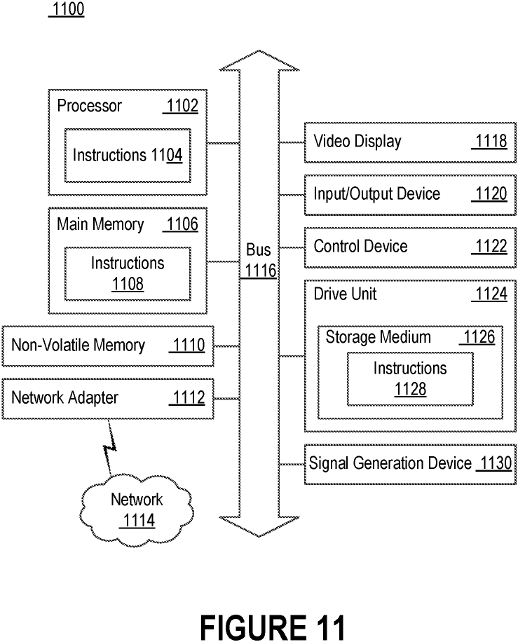

FIG. 11 is a block diagram illustrating an example of a system 1100 (e.g., the computing system 110 or a portion thereof, such as the processing system 112) in accordance with one or more implementations of the present technology. For example, some components of the system 1100 may be hosted on a computing device that includes a mutation analysis mechanism and a refinement mechanism.

The system 1100 may include a processor 1102, main memory 1106, non-volatile memory 1110, network adapter 1112, video display 1118, input/output device 1120, control device 1122 (e.g., a keyboard or pointing device), drive unit 1124 including a storage medium 1126, and signal generation device 1130 that are communicatively connected to a bus 1116. The bus 1116 is illustrated as an abstraction that represents one or more physical buses or point-to-point connections that are connected by appropriate bridges, adapters, or controllers. The bus 1116, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), inter-integrated circuit (I²C) bus, or an Institute of Electrical and Electronics Engineers (IEEE) standard 1394 bus (also referred to as “Firewire”).

While the main memory 1106, non-volatile memory 1110, and storage medium 1126 are shown to be a single medium, the terms “machine-readable medium” and “storage medium” should be taken to include a single medium or multiple media (e.g., a centralized/distributed database and/or associated caches and servers) that store one or more sets of instructions 1128. The terms “machine-readable medium” and “storage medium” shall also be taken to include any medium that is capable of storing, encoding, or carrying a set of instructions for execution by the system 1100.

In general, the routines executed to implement the implementations of the disclosure may be implemented as part of an operating system or a specific application, component, program, object, module, or sequence of instructions (collectively referred to as “computer programs”). The computer programs typically comprise one or more instructions (e.g., instructions 1104, 1108, 1128) set at various times in various memory and storage devices in a computing device. When read and executed by the processors 1102, the instruction(s) cause the system 1100 to perform operations to execute elements involving the various aspects of the present disclosure.

Further examples of machine- and computer-readable media include recordable-type media, such as volatile memory devices and non-volatile memory devices 1110, removable disks, hard disk drives, and optical disks (e.g., Compact Disk Read-Only Memory (CD-ROMS) and Digital Versatile Disks (DVDs)), and transmission-type media, such as digital and analog communication links.

The network adapter 1112 enables the system 1100 to mediate data in a network 1114 with an entity that is external to the system 1100 (e.g., between the processing system 112 can the sourcing device 152) through any communication protocol supported by the system 1100 and the external entity. The network adapter 1112 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### Remarks

The foregoing description of various implementations of the claimed subject matter has been provided for the purposes of illustration and description. It is not intended to be exhaustive or to limit the claimed subject matter to the precise forms disclosed. Many modifications and variations will be apparent to one skilled in the art. Implementations were chosen and described in order to best describe the principles of the invention and its practical applications, thereby enabling those skilled in the relevant art to understand the claimed subject matter, the various implementations, and the various modifications that are suited to the particular uses contemplated.

Although the Detailed Description describes certain implementations and the best mode contemplated, the technology can be practiced in many ways no matter how detailed the Detailed Description appears. Implementations may vary considerably in their implementation details, while still being encompassed by the specification. Particular terminology used when describing certain features or aspects of various implementations should not be taken to imply that the terminology is being redefined herein to be restricted to any specific characteristics, features, or aspects of the technology with which that terminology is associated. In general, the terms used in the following claims should not be construed to limit the technology to the specific implementations disclosed in the specification, unless those terms are explicitly defined herein. Accordingly, the actual scope of the technology encompasses not only the disclosed implementations, but also all equivalent ways of practicing or implementing the implementations.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various implementations is intended to be illustrative, but not limiting, of the scope of the technology as set forth in the following claims.

What is claimed is:

1. A system for processing a machine-learning (ML) model, the system comprising:
at least one processor; and
at least one memory coupled to the at least one processor and including processor instructions that, when executed by the at least one processor, perform operations including
accessing (i) a machine learning (ML) model to be trained to perform a given task and (ii) a dataset to be used for training purposes,
wherein the dataset includes text phrases that represent different DNA sequences associated with one or more types of cancer, and
wherein the ML model is configured to compute a cancer signature based on analyzing text phrases representative of patient DNA, the cancer signal signature representing (1) a likelihood that a corresponding patient has developed one or more types of cancer, (2) a likelihood that the patient will develop the one or more types of cancer within a given duration, (3) a development status at least leading up to onset or recurrence of the one or more types of cancer, or a combination thereof;
homomorphically encrypting at least the dataset;
providing the encrypted dataset to the ML model as input, so as to produce a trained ML model configured to process homomorphically encrypted data, wherein the trained ML model is configured to generate the cancer signature in ciphertext;
identifying, based on a computational architecture of the trained ML model, one or more computational locations at which to implement a noise reduction mechanism; and
programming the trained ML model such that the noise reduction mechanism is implemented at each identified computational location.

2. The system of claim 1, wherein the noise reduction mechanism includes a bootstrapping mechanism configured to re-encrypt the encrypted dataset according to homomorphic encryption.

3. The system of claim 2, wherein:
the dataset is associated with a key; and
the encrypted data set is re-encrypted using the key.

4. The system of claim 1, wherein the performed operations include:
calculating a refresh boundary within the trained ML model, wherein the refresh boundary represents a threshold number of allowable computations for maintaining reversible of noise levels; and
identifying a computational location within the refresh boundary, wherein the computation location is identified using a pseudo random selection mechanism and represents a timing for implementing the noise reduction mechanism.

5. The system of claim 4, wherein the refresh boundary is calculated based on a format of the dataset, a maximum estimated size of the dataset, a number of computational chains in the ML model, an allowable threshold for the homomorphic encoding mechanism, or a combination thereof.

6. The system of claim 1, wherein the trained ML model is programmed to iteratively implement the noise reduction mechanism at different times or locations to refresh results of processing the encrypted dataset over multiple iteration.

7. A method of operating a computing system, the method comprising:
receiving evaluation target data, wherein the evaluation target data includes ciphertext data representative of homomorphically encoded text phrases corresponding to patient DNA information;
selecting a machine learning (ML) model configured to compute a cancer signature based on analyzing text phrases representative of DNA information, the cancer signal signature representing (1) a likelihood that a corresponding patient has developed one or more types of cancer, (2) a likelihood that the patient will develop the one or more types of cancer within a given duration, (3) a development status at least leading up to onset or recurrence of the one or more types of cancer, or a combination thereof;
implementing the ML model using the evaluation target data as an input to test the evaluation target data against the ML model, wherein implementing the ML model includes generating the cancer signal signature in ciphertext based on:
iteratively performing cancer-evaluation computations using the encoded evaluation target data;
determining a trigger timing for implementing a noise reduction mechanism according to a progress of performing the cancer-evaluation computations;
implementing the noise reduction mechanism during the iterative computations according to the trigger timing, wherein the noise reduction mechanism is configured to remove internally-generated noise resulting from processing the encoded evaluation target data; and
communicating the ciphertext cancer signal signature to an external interface for decrypting the cancer signal signature with additional authorization information.

8. The method of claim 7, wherein the noise reduction mechanism includes a bootstrapping mechanism configured to re-encrypt the encrypted dataset according to the homomorphic encoding.

9. The method of claim 8, further comprising:
receiving a key or a derivative thereof, wherein the key was used to initially encode the evaluation target data,
wherein implementing the noise reduction mechanism includes re-encrypting the encrypted data set using the key or the derivative thereof.

10. The method of claim 7, further comprising:
determining a refresh boundary within the trained ML model, wherein the refresh boundary represents a threshold number of allowable computations for maintaining reversible of noise levels; and
identifying a computational location within the refresh boundary, wherein the computation location is identified using a pseudo random selection mechanism and represents a timing for implementing the noise reduction mechanism.

11. The method of claim 10, wherein the refresh boundary, the computational location, or a combination thereof are preset within the selected ML model.

12. The method of claim 10, wherein the refresh boundary, the computational location, or a combination thereof are dynamically computed after receiving the evaluation target data.

13. The method of claim 10, wherein the pseudo random selection mechanism includes a linear feedback shift registers (LFSR) based on prime polynomials.

14. The method of claim 7, wherein implementing the noise reduction mechanism includes iteratively implementing the noise reduction mechanism at different times or locations, each implementation of the noise reduction mechanism for removing noise corresponding to a portion of the evaluation target data and a combination of the implementations for removing noise from an entirety of the evaluation target data.

15. The method of claim 7, wherein the selected ML model includes a dummy operation that is reversed during subsequent computation, wherein the dummy operation is configured to create false computation paths for increasing privacy protection of the evaluation target data.

16. A non-transitory medium with instructions stored thereon that, when executed by a processor of a computing device, cause the computing device to perform operations comprising:
receiving evaluation target data, wherein the evaluation target data includes ciphertext data representative of homomorphically encoded text phrases corresponding to patient DNA information; and
implementing a machine learning (ML) model using the evaluation target data as an input to test the evaluation target data against the ML model,
wherein the ML model is configured to compute a cancer signature based on analyzing text phrases representative of DNA information,
wherein implementing the ML model includes implementing the noise reduction mechanism configured to remove internally-generated noise resulting from processing the encoded evaluation target data, and
wherein the computed cancer signal signature represents (1) a likelihood that a corresponding patient has developed one or more types of cancer, (2) a likelihood that the patient will develop the one or more types of cancer within a given duration, (3) a development status at least leading up to onset or recurrence of the one or more types of cancer, or a combination thereof.

17. The non-transitory medium of claim 16, wherein the noise reduction mechanism includes a bootstrapping mechanism configured to re-encrypt the encrypted dataset according to homomorphic encryption.

18. The non-transitory medium of claim 16, wherein implementing the noise reduction mechanism includes:
calculating a refresh boundary within the trained ML model, wherein the refresh boundary represents a threshold number of allowable computations for maintaining reversible of noise levels; and
identifying a computational location within the refresh boundary, wherein the computation location is identified using a pseudo random selection mechanism and represents a timing for implementing the noise reduction mechanism.

19. The non-transitory medium of claim 16, wherein implementing the noise reduction mechanism includes iteratively implementing the noise reduction mechanism at different times or locations, each implementation of the noise reduction mechanism for removing noise corresponding to a portion of the evaluation target data and a combination of the implementations for removing noise from an entirety of the evaluation target data.

20. The non-transitory medium of claim 16, wherein implementing the ML model includes executing a dummy operation that is reversed during subsequent computation, wherein the dummy operation is configured to create false computation paths for increasing privacy protection of the evaluation target data.
